(19) United States
(12) Patent Application Publication
(10) Pub. No.: US 2010/0099203 A1
(43) Pub. Date: Apr. 22, 2010

(54) Substrates with surfaces modified with PEG

(75) Inventors: Daniel Chang, Danville, CA (US); Scot Weinberger, Montara, CA (US)

Correspondence Address: QUINE INTELLECTUAL PROPERTY LAW GROUP, P.C., P O BOX 458, ALAMEDA, CA, 94501, US

(73) Assignee: Molecular Sensing, Inc., Montara, CA (US)

(21) Appl. No.: 12/587,112

(22) Filed: Sep. 30, 2009

Related U.S. Application Data
(60) Provisional application No. 61/102,750, filed on Oct. 3, 2008.

(51) Int. Cl. G01N 33/554; B32B 17/06; B32B 13/04; B32B 9/04; B32B 15/00; B32B 3/02; C12N 5/071; B05D 3/00; B32B 37/02; G01N 33/551; G01N 33/552; G01N 33/544; G01N 33/549

(52) U.S. Cl. 436/519; 428/426; 428/446; 428/411.1; 428/702; 428/689; 428/704; 428/172; 435/325; 428/336; 427/331; 156/325; 156/336; 436/524; 436/527; 436/528; 436/532

(57) ABSTRACT

Compositions and methods of modifying surfaces using hydroxyl terminated PEG are described herein. The surfaces so modified are useful in detection of synthetic and natural product organic molecules, organometallics, biomolecules, particles, or cells.

### CROSS-REFERENCE

This application claims the benefit of the filing date of U.S. provisional application 61/102,750, filed Oct. 3, 2008.

### BACKGROUND OF THE INVENTION

Detection of biological molecules, organic and synthetic molecules, particles and cells is an ever increasing field of technical complexity and commercial demand. It is desirable to detect molecules, particles and cells of interest at low concentration in-vitro and in short time periods. Therefore, there is a need to develop novel modified substrates capable of being used in in-vitro assay formats which exhibit lower levels of nonspecific binding and may offer increased precision of detection.

Additionally, the requirement for reduction of nonspecific binding on the substrate within the detection zone also applies to the fluidic conduits which conduct the sample to the detection zone, fluidic handling apparatus such as micropipettes and other dispensing means, and any of the components of bioanalytical apparatus that contacts such biological molecules, organic and synthetic molecules, particles and cells.

The reduction of nonspecific binding is also needed for other materials used in a biological environment such as prostheses, stents, sutures, shunts, etc, or for the handling of materials of biological materials ex-vivo such as tubing, containments, or transfer apparatus.

### SUMMARY OF THE INVENTION

A surface modification which provides such reduction of nonspecific binding is described herein. It utilizes a monolayer of polymer which has terminal hydroxy groups, which is covalently attached to a substrate.

In one aspect, the invention provides an article which comprises a substrate comprising a surface, and a PEG monolayer attached to the surface through a covalent linkage, wherein a plurality of PEG moieties comprise terminal hydroxy groups.

In another aspect, the invention provides an article which comprises a substrate comprising at least one channel having a surface wherein PEG is attached to the surface as a monolayer and a plurality of PEG moieties comprise a free terminal hydroxy group.

In another aspect, the invention provides an article which comprises a channel having a passivated surface comprising terminal hydroxy groups that exhibits substantially no nonspecific binding.

In another aspect, the invention provides an article which comprises a substrate having a microfluidic channel having a passivated surface comprising terminal hydroxy groups wherein the surface comprises a monolayer of a polymer of no more than 20 monomeric units.

In another aspect, the invention provides a method of passivating a surface which comprises the steps of reacting the surface with a reagent bearing reactive groups at both termini to produce the modified surface having a terminal reactive group; and reacting PEG comprising two terminal hydroxy groups with the terminal reactive group to attach PEG to the surface through a covalent bond to form a passivated surface comprising PEG which comprises terminal hydroxy groups.

In another aspect, the invention provides a method of manufacturing an article comprising two pieces sandwiched together, wherein at least one piece comprises a groove that defines the channel in the sandwich and wherein the two pieces each comprise a surface comprising a PEG monolayer comprising terminal hydroxy groups attached to the surface through a covalent linkage, which comprises the steps of attaching a reactive functionality to at least one surface comprising a PEG monolayer which comprises terminal hydroxy groups, and bonding both surfaces together by reacting the functionality to the PEG monolayer comprising terminal hydroxy groups on the surface of the second piece.

In another aspect, the invention provides a method which comprises the steps of providing a substrate comprising a glass surface having a PEG monolayer comprising terminal hydroxy groups attached thereto, wherein a portion of the terminal hydroxy groups of the PEG comprises terminal reactive functionalities; and reacting the terminal reactive functionalities with binding moieties to couple the binding moieties to the surface.

In another aspect, the invention provides a method which comprises the steps of providing a chip comprising a microfluidic channel therein defined by a wall, wherein the wall is passivated with a monolayer of PEG which comprises terminal hydroxy groups; flowing a fluid through the channel, wherein the fluid comprises a pair of binding partners; and detecting binding of the binding partners in the channel.

In some embodiments, the surface of the article of the invention is glass, silicon, silicone, polymer, ceramic, metal-oxide or metallic. In some embodiments, the reagent bearing reactive groups at both termini is toluene diisocyanate. In some embodiments, the terminal reactive group is an isocyanate group. In some embodiments of the invention, a polymer is covalently attached to the surface. In some embodiments of the invention, the covalent linkage is a urethane linkage. In some embodiments of the invention, the polymer is PEG.

In some embodiments of the invention, the PEG monolayer comprising terminal hydroxy groups is a brush monolayer. In some embodiments, the PEG monolayer comprising terminal hydroxy groups is a self assembled monolayer (SAM). In some embodiments of the invention, the PEG is PEG 2-50mer. In some embodiments of the invention, the PEG is PEG 2-20mer. In some embodiments of the invention, the PEG is PEG-200. In some embodiments of the invention, the PEG is PEG 5mer. In some embodiments of the invention, the PEG is substantially monodispersed. In some embodiments of the invention, the PEG is at least 95% monodispersed. In some embodiments, the monolayer is no more than 100 Ångstroms thick

In some embodiments of the invention, the surface is passivated with a monolayer of PEG comprising terminal hydroxy groups. In some embodiments of the invention, the passivated surface exhibits at least 95% reduction of nonspecific binding compared to a nonpassivated surface.

In some embodiments of the invention, a plurality of PEG moieties comprise a reactive functionality coupled through a terminal hydroxy group. In some embodiments of the invention, a plurality of terminal hydroxy groups of the PEG are coupled to a reactive functionality. In some embodiments of the invention, the reactive functionality is an epoxide, N-hydroxy succinimide (NHS), N-hydroxymaleimide (NHM), imidazolecarbonyl, hydrazine, aldehyde, iodoacetyl, cystamine, or dithiothreitol (DTT) group. In some embodiments of the invention, the reactive functionality is an epoxide.

In some embodiments of the invention, the reactive functionality is further coupled to a binding moiety. In some embodiments of the invention, the binding moiety is coupled through reaction with the reactive functionality. In some embodiments of the invention, the binding moiety is a biomolecule. In some embodiments of the invention, biomolecule is an antibody, nucleic acid, organic molecule, protein, particle, or cell. In some embodiments of the invention, the organic molecule is a hapten, receptor ligand, phosphatidyl glycol, avidin, biotin, organometallic, drug molecule, aptamer, or heparin.

In some of the embodiments of the invention, detection of the binding partners is by interferometry.

In some embodiments, the article of the invention comprises a substrate comprising at least one channel. In some embodiments, the channel is a microfluidic channel. In some embodiments, the article of the invention comprises a substrate which comprises two pieces sandwiched together, wherein at least one piece comprises a groove that defines the channel in the sandwich. In some embodiments, the channel is a microfluidic channel having a diameter no greater than 500 microns. In some embodiments, the channel is a microfluidic channel having an average cross sectional area of about 0.2 mm². In some embodiments, the at least one channel is at least two channels.

In some of the embodiments of the methods of the invention, the bonding of both surfaces is at room temperature. In some of the embodiments of the methods of the invention, the reactive functionality is epoxide, acrylate, or methacrylate.

In another aspect this invention provides a method comprising: (a) providing a chip comprising a channel therein defined by a wall, wherein the wall is passivated with a monolayer of PEG comprising terminal hydroxy groups and comprising a binding moiety attached to the wall; (b) flowing a fluid through the channel, wherein the fluid comprises a binding partner for the binding moiety; and (c) capturing the binding partner with the binding moiety. In one embodiment the channel is a microfluidic channel. In another embodiment the binding moiety is a biomolecule. In another embodiment the biomolecule is an antibody, nucleic acid, organic molecule, protein, particle, or cell. In another embodiment the organic molecule is a hapten, receptor ligand, phosphatidyl glycol, avidin, biotin, organometallic, drug molecule, or heparin. In another embodiment the method further comprises detecting binding of the binding partner in the channel. In another embodiment binding is detected by interferometry. In another embodiment the effective concentration of detectable binding of the binding partners is lower than 100 pM. In another embodiment the effective concentration of detectable binding of the binding partners is lower than 100 pM.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic representation of the manufacturing process to create a passivated surface of the invention.

FIG. 2 is a schematic representation of the topology of a PEG-200 passivated surface and a PEG-5mer passivated surface.

FIG. 3 is a schematic representation of the manufacturing process to create a preactivated PEG passivated surface of the invention which has epoxy groups as binding moieties.

FIG. 4 is a graphical representation of the AFM pictogram of a PEG-200 passivated Borofloat 33 chip.

FIG. 5 is a graphical representation of the AFM pictogram of a PEG-200 passivated silicon chip.

FIG. 6A is a graphical representation of the FTIR spectrum of a PEG-200 passivated fluidic chip used in Back Scattering Interferometry (BSI) measurements.

FIG. 6B is a graphical representation of the FTIR spectrum of a PEG-200 passivated aluminum fluidic chip used in Back Scattering Interferometry (BSI) measurements.

FIG. 7 is a graphical representation of the ellipsometry characterization and modeling of a PEG-200 brush film thickness.

FIG. 8 is a three dimensional representation of an AFM scan of a PEG-5mer passivated silicon surface.

FIG. 9A is a two dimensional representation of the surface roughness from an AFM scan of a PEG-5mer passivated silicon surface.

FIG. 9B is a one dimensional representation of the roughness analysis of an AFM scan of a PEG-5mer passivated silicon surface.

FIG. 10 is a graphical representation of a Fourier Transform Infrared Spectrum of a PEG-5mer passivated alumina surface.

FIG. 11 is a graphical representation of a section of the FTIR Spectrum shown in FIG. 10.

FIG. 12 is a graphical representation of the ellipsometry characterization and modeling of a PEG-5mer brush film thickness.

FIG. 13 is a schematic representation of the interaction between a protein and a PEG-5mer passivated surface of the invention.

FIG. 14 is a schematic representation of the interaction between a protein and an epoxy activated PEG-5mer passivated surface of the invention.

FIG. 15A is a graphical representation of the FTIR spectrum for a PEG-5mer passivated alumina surface.

FIG. 15B is a graphical representation of the FTIR spectrum for an activated epoxy functionalized PEG-5mer passivated alumina surface.

FIG. 15C is a graphical representation of the subtractive FTIR spectrum showing the absorbance peaks due to the epoxy functionalities on an activated epoxy functionalized PEG-5mer passivated alumina surface.

FIG. 16A is a graphical representation of the FTIR spectrum for a PEG-5mer passivated glass surface.

FIG. 16B is a graphical representation of the FTIR spectrum for an activated epoxy functionalized PEG-5mer passivated glass surface.

FIG. 16C is a graphical representation of the subtraction FTIR spectrum showing the absorbance peaks due to the epoxy functionalities on an activated epoxy functionalized PEG-5mer passivated glass surface.

FIG. 17 is a pictorial representation of channels in PEG-200 passivated glass chip B5 and of channels in unmodified glass chip B2. Panel A represents the PEG-200 passivated glass chip B5 channel under normal light. Panel D represents the unmodified glass chip B2 channel under normal light. Both chips were incubated with 1 mg/ml of Protein A-FITC for 10 minutes at RT, and washed with DI water. Panel B represents the PEG-200 passivated glass chip B5 channel image after 5 second of exposure (fluorescent intensity collection). Panel C represents the PEG-200 passivated glass chip B5 channel image after 10 seconds of exposure Panel E represents the unmodified glass chip B2 channel image after 5 sec of exposure. Panel F represents the unmodified glass chip B2 channel image after 10 seconds of exposure.

FIG. 18 is a graphical representation of the quantification of the difference in fluorescence intensity observed between a PEG-200 passivated glass chip B5 channel exposed to a fluorescent protein and an unmodified glass chip B2 channel exposed to the same fluorescent protein as a measure of nonspecific binding.

FIG. 19 is a graphical representation of the performance evaluation by glycerol calibration of a PEG-200 passivated glass Back Scattering Interferometry (BSI) chip B5 channel compared to an unmodified glass chip B2 channel.

FIG. 20A is a graphical representation of the determination of the KD of binding between IgG and Protein A using Back Scattering Interferometry on a PEG-200 passivated chip.

FIG. 20B is a graphical representation of the determination of the KD of binding between IgG and Protein A using Back Scattering Interferometry on a PEG 5-mer passivated chip.

FIG. 21 shows a flow diagram for backscattering interferometry.

FIG. 22 shows a Fourier transform algorithm that transforms a digital image into a function that describes the image.

FIG. 23 shows a flow chart for the Gaussian fit analysis.

FIG. 24 shows the use of a hamming window.

FIG. 25 shows a system for sample analysis in flowing streams.

### DETAILED DESCRIPTION OF THE INVENTION

### 1. Substrates with Surfaces Passivated with a PEG Monolayer

1.1. Introduction

This invention provides substrates having surfaces passivated with a monolayer of PEG. As referred to herein, passivate is used to mean that nonspecific adsorption of biological or synthetic molecules to such passivated surface is reduced. In particular, the PEG used to passivate the surface can be a short PEG polymer, for example having length between about 2 and about 50 monomers in length. The PEG can have average mass of about 200 daltons (e.g., PEG 200). The PEG can have length of about 5 monomeric units. The PEG can be substantially monodisperse. The PEG can be attached without protection at one terminus, so the surface comprises PEG moieties comprising a free terminal hydroxyl group. At least a portion of the PEG moieties comprise free terminal hydroxyl groups. In some embodiments, a majority, substantially all or all of the PEG moieties comprise free terminal hydroxyl groups.

The substrate can be any material comprising a surface amenable to modification by the attachment of PEG. In certain embodiments, the surface comprises hydroxyl groups. These groups can be reacted with bifunctional agent, such as a diisocyanate. The remaining active group can be reacted with PEG to attach it to the surface. In certain embodiments, the PEG comprises free terminal hydroxyl groups. These groups are, themselves, useful as points of attachment of binding moieties, such as antibodies, receptors, nucleic acids and the like. In this way, the surface comprises a PEG monolayer and attached binding moieties. The binding moieties can, in turn, be used capture their binding partners from a sample placed in contact therewith.

In the methods of the invention, compositions are described which decrease the nonspecific binding often observed when molecules, particles or cells come in contact with substrate surfaces and which therefore provide novel modified substrates for use in analysis of molecules, particles, or cells. These modified surfaces are passivated with respect to nonspecific binding by molecules, particles, or cells. Nonspecific binding occurs through a variety of basic molecular-level adhesion mechanisms, including all combinations of electrostatic, hydration, hydrophobic, acid-base, dispersive and hydrogen bonding interactions. Such nonspecific binding can not only interfere with the precision of the detection, if molecules, particles, or cells other than the desired target molecule, particle, or cell binds to the substrate surface, but such nonspecific binding to the substrate surface may also alter the three dimensional characteristics or chemical behavior of the target molecule, particle, or cell, which in turn may alter the detection or analysis of the target molecule, particle, or cell sufficiently to render such detection or analysis unreliable. Further, nonspecific binding of the target molecule, particle or cell within the sample introduction zone upstream of a detection zone reduces the number of entities which are detected, with potentially negative impact on accuracy or precision, even so far as to reduce the numbers of detectable entities below the limits of detection of the instrument.

In a second aspect of the invention, the passivated surfaces of the invention are useful in a wide range of other applications besides bioanalysis. Surfaces that have been modified to reduce nonspecific binding of molecules, particles or cells may be utilized in vessels, tubing and transfer apparatus which contact materials of many types including foods, biological fluids, implantable devices or specialized manufacturing or transfer of hydrophobic materials.

While nonspecific binding to surfaces is most often undesirable, specific capture of designated biomolecules, drugs, synthetic molecules, particles, or cells by binding at a target surface is often desirable. Examples include the specific binding of bioactive antibodies on a surface for immunoassay applications, specific binding of nucleic acid primers on a surface for genetic assays (DNA and protein microarrays or microfluidic assays, for example, and including DNA microarrays), and specific binding of receptors, growth factors, or antibiotics, for example, to surfaces for protein microassays, including, for example, protein microassays. Such specific binding to a surface binds one or a limited number of designated types of molecules, particles, or cells to the substrate surface and does so in a manner that preserves the recognition activity and native structure and function of the specifically bound molecule, particle or cell.

Thus, functional surface chemistries are needed that (1) inhibit nonspecific binding of unwanted molecules, particles or cells to a treated surface; (2) inhibit nonspecific binding of unwanted molecules, particles, or cells to a treated surface while promoting specific molecule, particle or cells binding to that same surface; or (3) that inhibit nonspecific binding of unwanted molecules, particles, or cells to a treated surface while promoting functionally specific and/or biologically active molecule, particle, or cell binding, to that same surface.

Substrate surfaces are often modified by covalently binding biomolecules or organic molecules to the surface of the substrate to change its surface characteristics. These molecules are selected to modulate the electrostatic, hydration, hydrophobic, acid-base, dispersive and/or hydrogen bonding characteristics of the unmodified substrate surface, thus passivating the surface with respect to nonspecific binding of target molecules, particles, or cells. These molecules are often polymers, which may be PEGS (oligomers or polymers), polyethylenimines, polystyrene, polysiloxanes, polyurethanes, proteins, poly(amino acids), polyphosphazenes, telechelic block copolymers (Pluronics™), polyacrylates, polyacrylamides, polymethacrylates, polysaccharides, dendrimers, macromonomers, or heterobifunctional alkyl-linked coupling reagents.

However, these species, in some embodiments, are not reactive enough to couple directly to the substrate surface and an initial surface modification may be performed with a more reactive species which can both react with the surface of the substrate directly and also react with the chemical species which will provide the desired modulation of nonspecific binding and/or induction of binding of the target molecule, particle, or cell.

Therefore, a modified substrate surface suitable for use in detection of molecules, particles, or cells is produced by initially reacting one reactive group of a reagent bearing two reactive groups to the substrate surface; reacting the remaining reactive group on the reagent with a polymer to introduce surface modification which will modulate nonspecific binding; and optionally reacting a remaining terminal group with another chemical species which includes or permits further introduction of a reactive moiety. This reactive moiety can specifically couple a binding moiety to the surface via the surface modifying polymer. The resultant modified substrate is used to detect target molecules. In some embodiments, the modified substrate does not have a binding moiety attached to the substrate to capture the target molecules. In some embodiments, the modified substrate has a binding moiety attached to the substrate as described above, which binds to a binding partner which is a molecule, particle, or cell of interest. The target molecules, particles, or cells are detected, without substantial nonspecific binding of target or non-target molecules, particles, or cells. Monodisperse compositions are particularly useful in articles wherein the surfaces are both passivated and comprise binding functionalities.

1.2. Substrates

The substrates of this invention include any solid material. Substrates typically comprise a surface capable of being coated with the combination of components of the present invention. Suitable substrates include refractive, transparent, adsorptive and opaque solid-phase materials. They include, but are not limited to metals, such as, gold, platinum, silver, copper, iron, aluminum; polymers: such as polystyrene, polysulfone, polyetherimide, polyethersulfone, polysiloxane, polyester, polycarbonate, polyether, polyacrylate, polymethacrylate, cellulose, nitrocellulose, perfluorinated polymers, polyurethane, polyethylene, polyamide, polyolefin, polypropylene, nylon, hydrogels, and related blends and copolymers; non-metals, such as silica, silicon dioxide, titanium oxides, aluminum oxides, iron oxides, carbon, silicon, silicon nitride, various silicates and other glasses, for example soda-lime glass, ceramics and sol-gels. In some embodiments, the substrate is hydrophilic. In some embodiments, the substrate is hydrophobic.

In some embodiments of the invention, the substrate is a non-metal, such as silica. In some embodiments of the invention, the substrate is a non-metal, such as silicon dioxide. In some embodiments of the invention, the substrate is a non-metal, such as titanium oxides. In some embodiments of the invention, the substrate is a non-metal, such as aluminum oxides, iron oxides. In some embodiments of the invention, the substrate is a non-metal, such as carbon. In some embodiments of the invention, the substrate is a non-metal, such as silicon. In some embodiments of the invention, the substrate is a non-metal, such as carbon. In some embodiments of the invention, the substrate is a non-metal, such as silicon nitride. In some embodiments of the invention, the substrate is a non-metal, such as a silicate. In some embodiments of the invention, the substrate is a non-metal, such as a glass. In some embodiments of the invention, the substrate is a non-metal, such as soda-lime glass. In some embodiments of the invention, the substrate is a non-metal, such as a ceramic. In some embodiments of the invention, the substrate is a non-metal, such as a sol-gel.

In some embodiments of the invention, the substrate is gold metal. In some embodiments of the invention, the substrate is platinum metal. In some embodiments of the invention, the substrate is silver metal or its oxidized forms. In some embodiments of the invention, the substrate is copper metal or its oxidized form. In some embodiments of the invention, the substrate is iron metal or its oxidized form. In some embodiments of the invention, the substrate is aluminum metal or its oxidized form.

In some embodiments of the invention, the substrate is a polymer, such as polystyrene. In some embodiments of the invention, the substrate is a polymer, such as polysulfone. In some embodiments of the invention, the substrate is a polymer, such as polyetherimide. In some embodiments of the invention, the substrate is a polymer, such as polyethersulfone. In some embodiments of the invention, the substrate is a polymer, such as polysiloxane. In some embodiments of the invention, the substrate is a polymer, such as polyester. In some embodiments of the invention, the substrate is a polymer, such as polycarbonate. In some embodiments of the invention, the substrate is a polymer, such as polyether. In some embodiments of the invention, the substrate is a polymer, such as polyacrylate. In some embodiments of the invention, the substrate is a polymer, such as polymethacrylate. In some embodiments of the invention, the substrate is a polymer, such as cellulose. In some embodiments of the invention, the substrate is a polymer, such as nitrocellulose. In some embodiments of the invention, the substrate is a polymer, such as a perfluorinated polymer. In some embodiments of the invention, the substrate is a polymer, such as polyurethane. In some embodiments of the invention, the substrate is a polymer, such as polyethylene. In some embodiments of the invention, the substrate is a polymer, such as polyamide. In some embodiments of the invention, the substrate is a polymer, such as polyolefin. In some embodiments of the invention, the substrate is a polymer, such as polypropylene. In some embodiments of the invention, the substrate is a polymer, such as nylon. In some embodiments of the invention, the substrate is a polymer, such as a hydrogel and related blends and copolymers.

1.3. Surface Modification

In certain methods, surfaces are prepared for passivation with PEG by modifying the surfaces with reagents bearing reactive groups at both termini. One end is used to attach to the surface. The other end is used to attach to PEG.

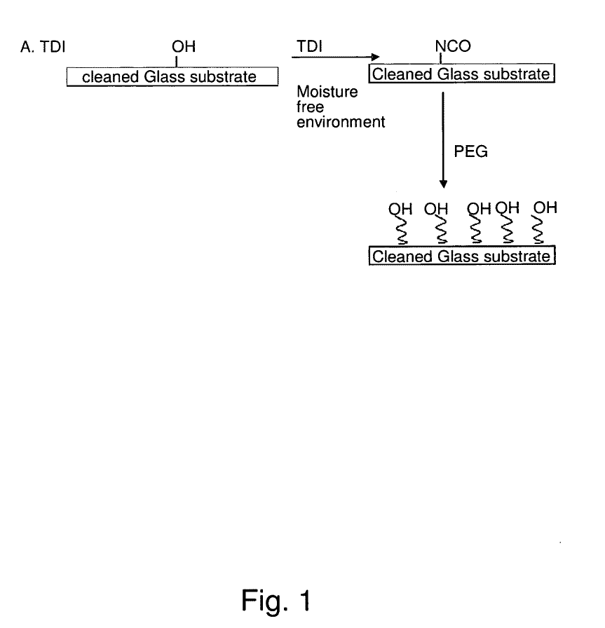

In some of the methods of the invention, it is possible to provide a self assembled monolayer (SAM), by dipping the substrate, having an already attached reagent bearing a reactive group at the remaining free terminus, into a composition containing PEG polymer. In other embodiments, a SAM is produced when the substrate has the composition containing PEG polymer flushed through one or more channels. In other embodiments of the invention, a SAM is produced when the initial modification of the substrate with a reagent bearing reactive groups at both termini and/or attachment step of introducing PEG to react with the remaining reactive group is performed under vapor deposition conditions. In some embodiments, this is a chemical vapor deposition (CVD) process, as shown schematically in FIG. 1. In this method, components are placed into a CVD oven. Vacuum is applied to remove all gas and to put the chamber at negative pressure. The oven is heated and gasses comprising the reactants are introduced. The reactants attach to all available surfaces of the substrate. Masks can be employed so that only selected surfaces are bonded with the reactants. This process can be repeated to achieve desired levels of coating. No other directed introduction of PEG is required to provide for efficient induction of the monolayer. In some embodiments, spatially directed introduction of reactive groups or PEG is performed by which means the reactive group or PEG reacts wherever it is spatially directed but still self assembles wherever it contacts the surface.

1.3.1. Reactive Moieties on the Substrate Surface

Substrates are modified by initially reacting chemical moieties on the substrate surface, which are amenable to chemical modification, with a reagent which bears reactive groups at both a first terminus and a second terminus, such that a reaction occurs between the chemically modifiable moieties on the substrate surface with a reactive group of either the first terminus or second terminus of the reagent.

For some embodiments of the invention, the chemical moieties on the substrate surface which are amenable to chemical modification are hydroxyl groups. For example, glass substrates present a population of hydroxyl groups. The density of these hydroxyl groups depends on the handling and storing conditions to produce the glass substrate. A number of other substrates also exhibit hydroxyl groups as usefully modifiable chemical moieties on the surfaces of those substrates. Examples include metals like aluminum and/or titanium, silica wafers, sol-gel and other polymeric substrates, some embodiments of ceramic substrates, and metal oxide substrates. Other chemically modifiable groups may include, but are not limited to sulfides, sulfhydryls, amino groups, boronates, carboxylates, and the like.

In some embodiments the chemically modifiable group on the substrate surface is a sulfide. In some embodiments the chemically modifiable group on the substrate surface is a sulfhydryl. In some embodiments the chemically modifiable group on the substrate surface is an amino group. In some embodiments the chemically modifiable group on the substrate surface is a boronate. In some embodiments the chemically modifiable group on the substrate surface is a carboxylate. In some embodiments the chemically modifiable group on the substrate surface is hydroxyl group.

The substrate is cleaned in preparation to applying a monolayer to it. In some embodiments, the cleaning step comprises chemical treatment which will induce the formation of chemically modifiable groups on the substrate surface, i.e. for example, the introduction of oxide groups on a metal surface.

In some embodiments the substrate is exposed to a solution of a reagent having two reactive termini and/or a solution of PEG comprising terminal hydroxy groups. In some embodiments the substrate is exposed to a vapor of a reagent having two reactive termini and/or a solution of PEG comprising terminal hydroxy groups. In some embodiments the substrate has a solution of a reagent having two reactive termini and/or a solution of PEG comprising terminal hydroxy groups flowed through one or more channels in the substrate. In some embodiments the substrate has a vapor of a reagent having two reactive termini and/or a solution of PEG comprising terminal hydroxy groups flowed through one or more channels in the substrate.

1.3.2. Reagents for Covalent Modification of the Substrate Surface

The reagent bearing reactive groups at both termini is used to functionalize the chemically modifiable groups already present on the substrate surface, by reacting at one of the two termini with the chemically modifiable group on the substrate surface, and retaining the second reactive group for further reaction with a polymer or other species that will act to modulate the nonspecific binding and/or be capable of further modification to induce specific binding of the target molecule, particle, or cell. The second reactive group still present is often more reactive than the chemically modifiable group on the original untreated surface and can therefore react with a wide variety of chemical species to introduce surface modification which will modulate nonspecific binding and/or be capable of inducing specific interactions of desired molecular species with the substrate.

Examples of the reagent bearing reactive groups at both termini to initially modify the substrate surface for use in the present invention include, but are not limited to, isocyanates (e.g., toluene diisocyanate), silanes, methacrylates, disulfides, disilazanes, sulfhydryls, acrylates, carboxylates, activated esters, other active leaving groups, isonitriles, phosphoamidites, nitrenes, epoxides, hydrosilyl, esters, arenes, azido, amine, nitrile, vinyl groups, alkylphosphonates, and other known surface-coupling reactive species known to those skilled in the art of chemical coupling to surfaces. In some embodiments, the reagent bearing reactive groups at both termini bears at least one silane group. In some embodiments, the reagent bearing reactive groups at both termini bears at least one methacrylate group. In some embodiments, the reagent bearing reactive groups at both termini bears at least one disulfide group. In some embodiments, the reagent bearing reactive groups at both termini bears at least one disilazane group. In some embodiments, the reagent bearing reactive groups at both termini bears at least one sulfhydryl group. In some embodiments, the reagent bearing reactive groups at both termini bears at least one acrylate group. In some embodiments, the reagent bearing reactive groups at both termini bears at least one carboxylate group. In some embodiments, the reagent bearing reactive groups at both termini bears at least one activated ester group. In some embodiments, the reagent bearing reactive groups at both termini bears at least one active leaving group. In some embodiments, the reagent bearing reactive groups at both termini bears at least one isonitrile group.

In some embodiments, the reagent bearing reactive groups at both termini bears at least one isocyanate group. In some embodiments, the reagent bearing reactive groups at both termini bears at least one phosphoramidite group. In some embodiments, the reagent bearing reactive groups at both termini bears at least one nitrene group. In some embodiments, the reagent bearing reactive groups at both termini bears at least one epoxide group. In some embodiments, the reagent bearing reactive groups at both termini bears at least one hydrosilyl group. In some embodiments, the reagent bearing reactive groups at both termini bears at least one ester group. In some embodiments, the reagent bearing reactive groups at both termini bears at least one arene group. In some embodiments, the reagent bearing reactive groups at both termini bears at least one azido group. In some embodiments, the reagent bearing reactive groups at both termini bears at least one amino group. In some embodiments, the reagent bearing reactive groups at both termini bears at least one nitrile group. In some embodiments, the reagent bearing reactive groups at both termini bears at least one vinyl group. In some embodiments, the reagent bearing reactive groups at both termini bears at least one alkylphosphonate group. The reactive groups at both termini may be the same or different.

In some embodiments the monolayer of polymer is attached to the substrate surface through a urethane bond, e.g., through reaction with an isocyanate. In some embodiments the monolayer of polymer is attached to the substrate surface through an amide bond. In some embodiments the monolayer of polymer is attached to the substrate surface through a carbamate bond. In some embodiments the monolayer of polymer is attached to the substrate surface through a guanidinium bond. In some embodiments the monolayer of polymer is attached to the substrate surface through an ether bond. In some embodiments the monolayer of polymer is attached to the substrate surface through a sulfide bond. In some embodiments the monolayer of polymer is attached to the substrate surface through a disulfide bond.

In some embodiments of the invention, isocyanate reagents are used to initially modify the substrate surface in preparation for covalently attaching a polymer, such as, for example, PEG having terminal hydroxy groups. One particularly useful species of this group of reagents is toluene diisocyanate (TDI). A schematic representation of this process is shown in FIG. 1.

1.3.3. Introduction of a PEG Monolayer to the Surface of the Substrate.

Many different polymers may be used to produce the modified surface of the substrate of the invention. In some embodiments the polymer is a polyethylene glycol (PEG) oligomer or polymer. Polyethylene glycol is a polymer formed from reacting ethylenediol units end-to-end, resulting in a polymer units which has two terminal hydroxy groups. Alternatively, the polymer can be a polyethylenimine, a polystyrene, a polysiloxane, a polyurethane, a protein, a poly(amino acid) or a polyphosphazene. These polymers are functionalized to contain hydroxyl functionalities at the terminus of the polymer opposite to the terminus which attaches to the surface via a covalent bond. In some embodiments the polymer is a telechelic block copolymer (Pluronics™). In some embodiments the polymer is a polyacrylate. In some embodiments the polymer is a polyacrylamide. In some embodiments the polymer is a polymethacrylate. In some embodiments the polymer is a polysaccharide. In some embodiments the polymer is a dendrimer. In some embodiments the polymer is a macromonomer. In some embodiments the polymer is a heterobifunctional alkyl-linked coupling reagent.

PEG can be used to modify the surface of the substrate and create a passivated surface which will have reduced nonspecific binding properties. In particular, PEG is attached to substrate surfaces to provide a monolayer of polymer. Only one terminus of PEG is coupled to the surface by the methods described herein. Multiple strands of the PEG polymer do not couple to each other during incorporation onto the passivated surface. Therefore a monolayer of controllable thickness is introduced and is determined by the length of the PEG polymer units used. One of the two hydroxy termini of the PEG polymer is covalently bonded to the surface and the second hydroxy termini is exposed to the environment above the substrate. This results in a monolayer which is attached to the surface at only one termini and not attached to other polymer units, yielding a monolayer with terminal hydroxy groups contacting the fluid environment which is untangled or crosslinked, referred to herein as a brush layer in that the polymer units resemble the bristles of a brush. In particular, the free terminal hydroxy groups of the immobilized PEG monolayer modulates the hydrophobicity/hydrophilicity of the substrate native surface to reduce nonspecific binding. In the PEG monolayers of the invention, the “brush-tip” of the brush layer, the termini of the monolayer in contact with the environment, is hydrophilic.

PEG is typically sold as polymer of dispersed molecular weights (polydisperse). That is, it comes as a mixture of polymers having different polymer lengths. A number associated with PEG typically indicates the average molecular weight of the composition, for example PEG-200 has an average molecular weight of about 200. In some embodiments of the invention, the PEG polymer used has a relatively narrow molecular weight/chain length distribution. For example, PEG 5-mer (pentaethylene glycol 98%), which is substantially only the polymer comprising five repeating units of ethylenediol.

In some embodiments of the invention, the PEG is a PEG of about 2-mer to about 50-mer units (PEG 2-50mer), about 2-mer to about 40-mer units (PEG 2-40mer), about 2-mer to about 30-mer units (PEG 2-30mer), about 2-mer to about 20-mer units (PEG 2-20mer), about 2-mer to about 10-mer units (PEG 2-10mer), about 2-mer to about 9-mer units (PEG 2-9mer), about 2-mer to about 8-mer units (PEG 2-8mer), about 2-mer to about 7-mer units (PEG 2-7mer), about 2-mer to about 6-mer units (PEG 2-6mer), about 2-mer to about 5-mer units (PEG 2-5mer), or about 2-mer to about 4-mer units (PEG₂₋₄). In other embodiments, 95% of the PEG polymers have lengths within 4 monomeric units, e.g., PEG 2-6mer or PEG 3-7mer. In any PEG composition, one species may be the predominant species, that is, the composition contains more of this species than any other species of PEG. So, for example, in a composition that is predominantly PEG 5-mer, there are more molecules of PEG 5-mer than any other species of PEG. Similarly, a composition that is predominantly PEG 200 comprises more PEG 200 than any other molecular weight of PEG. Monodisperse PEG is available from, for example, Polypure (Olso, Norway) (at least 95% pure). Monodisperse compositions of PEG also can be made by purification from polydisperse PEG using distillation and collection of the appropriate fraction containing the monodisperse compound (e.g., PEG 5mer (mw 258)).

In certain embodiments, the PEG used is a low molecular weight PEG. For example, the PEG can have an average length no greater than 20 monomeric units. The use of low molecular weight polymers, whether of narrow molecular weight distribution or substantially uniform weight distribution, may afford a more uniform monolayer, particularly as a brush monolayer, compared to substrate coatings which may not be controllably introduced as a monolayer, such as for example, silanol-modified substrate coatings.

In certain embodiments of this invention, PEG is monodispersed or substantially monodispersed. PEG is substantially monodispersed if at least 50% of the molecules in the mixture have the same molecular weight. In some embodiments, the monolayer of PEG is monodispersed, that is, in which the polymers all have the same length. In some embodiments the monolayer of PEG is more than any of: about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% monodispersed. In some embodiments of the invention, the monolayer of PEG is no less than any of: about 99%, about 98%, about 97%, about 96%, about 95%, about 94%, about 93%, about 92%, about 91%, about 90%, about 85%, about 80%, about 75%, about 70%, about 65%, about 60%, about 55%, or about 50% monodispersed.

The distribution of lengths of polymer chains, and hence, the distribution of molecular weight in a PEG polymer population is represented by several parameters. The weight average molecular mass (M_{w}) is which is a weighted mean using molecular weight (M_{w}=ΣMᵢ²Nᵢ/ΣMᵢNᵢ) and the number average molecular mass (Mₙ) is a weighted mean using molecular fraction (Mₙ=ΣMᵢNᵢ/ΣNᵢ). The polydispersity of a polymer population is reflected in the Polydispersity Index (PDI) which is equal to M_{w}/Mₙ, and reflects how narrow or broad the weight/chain length distribution is in the polymer population.

M_{w}=ΣMᵢ²Nᵢ/ΣMᵢNᵢ (Eq. 1)

Mₙ=ΣMᵢNᵢ/ΣNᵢ (Eq. 2)

PDI=M_{w}/Mₙ (Eq. 3)

In some embodiments, the PEG has a PDI of about 1.0, about 1.1, about 1.2, about 1.3, about 1.4, about 1.5, about 1.7, about 1.8, about 1.9, about 2.0, about 2.2, about 2.4, about 2.5, about 3.0, about 4.0. about 5.0 or about 6.0. The PEG can have a PDI range of 1 to 6.

In some embodiments of the invention, surface homogeneity is required so that the monolayer does not interfere with measurements using Back Scattering Interferometry (BSI), a measurement of which is dependent on the use of coherent, monochromatic light sources with minimal optical scatter and dispersion in the detection zone. In some embodiments the monochromatic light source is a helium-neon laser with a wavelength of about 633 nm. In order to constrain optical scatter and dispersion, in some embodiments the variation in surface homogeneity of the detection zone is no more than any of: about λ/10, about λ/20, about λ/30, about λ/40, about λ/50, about λ/60, about λ/70, about λ/80, about λ/90, about λ/100, about λ/110, about λ/120, about λ/130, about λ/140, about λ/150, or about λ/160, where λ is the wavelength of the coherent, monochromatic source.

In some embodiments the, thickness of the PEG monolayer comprising terminal hydroxy groups is about λ/30, about λ/40, about λ/50, about λ/60, about λ/70, about λ/80, about λ/90, about λ/100, about λ/110, about λ/120, about λ/130, about λ/140 or about λ/150.

In some of the embodiments the PEG has a median molecular weight of about 100 Daltons, about 110 Daltons, about 120 Daltons, about 130 Daltons, about 140 Daltons, about 150 Daltons, about 160 Daltons, about 170 Daltons, about 180 Daltons, about 190 Daltons, about 200 Daltons, about 210 Daltons, about 220 Daltons, about 230 Daltons, about 240 Daltons, about 250 Daltons, about 260 Daltons, about 270 Daltons, about 280 Daltons, about 290 Daltons, about 300 Daltons, about 310 Daltons, about 320 Daltons, about 330 Daltons, about 340 Daltons, about 350 Daltons, about 360 Daltons, about 370 Daltons, about 380 Daltons, about 390 Daltons, about 400 Daltons, about 400 Daltons, about 450 Daltons, about 500 Daltons, about 550 Daltons, about 600 Daltons, about 650 Daltons, about 700 Daltons, about 750 Daltons, about 800 Daltons, about 900 Daltons, or about 1000 Daltons.

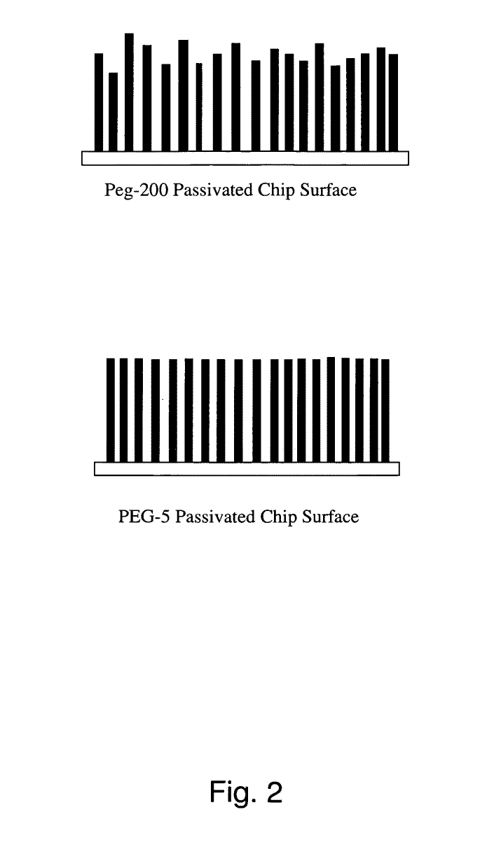

In one embodiment of the invention, a terminal hydroxyl group of PEG-200 is reacted with the isocyanate modified surface of a substrate. PEG-200 is a mixture of polyethylene glycol monomers, with a molecular weight distribution of about 180 to about 250. The shorter length of polymer chain in the brush monolayer reduces the potential for self association, folding, and complicated surface topology at the modified surface. The relatively narrow weight distribution, and hence, chain length distribution, may provide a more uniform monolayer than, for example, a PEG distribution with a far greater weight distribution. It may be used in an embodiment which is a self assembled monolayer (SAM). A schematic representation is shown in FIG. 2.

In another embodiment of the invention, PEG 5-mer is used (i.e., PEG with 5 monomeric units). It is a specific composition of polyethylene glycol, having substantially only five repeat units, and thus, with a uniform weight of 238 Daltons. It is coupled through a terminal hydroxyl group in order to attach it to a substrate surface. The brush monolayer which is formed is more uniform than brush monolayers formed by PEG populations with a weight distribution rather than a specific molecular weight. A schematic representation is shown in FIG. 2. PEG 5mer may also be used to produce a self assembling monolayer. PEG 5mer can be the predominate species in the mixture.

In some embodiments the PEG is a PEG of about 2 monomer units, about 3 monomer units, about 4 monomer units, about 5 monomer units, about 6 monomer units, about 7 monomer units, about 8 monomer units, about 9 monomer units, about 10 monomer units, about 11 monomer units, about 12 monomer units, about 13 monomer units, about 14 monomer units, about 15 monomer units, about 16 monomer units, about 17 monomer units, about 18 monomer units, about 19 monomer units, about 20 monomer units, about 21 monomer units, about 22 monomer units, about 23 monomer units, about 24 monomer units, about 25 monomer units, about 26 monomer units, about 27 monomer units, about 28 monomer units, about 29 monomer units, about 30 monomer units, about 31 monomer units, about 32 monomer units, about 33 monomer units, about 34 monomer units, about 35 monomer units, about 36 monomer units, about 37 monomer units, about 38 monomer units, about 39 monomer units, or about 40 monomer units.

In some of the embodiments of the invention, the surface of the substrate that is modified with a PEG monolayer which comprises terminal hydroxy groups will be used as part of an article without additional modification. In some embodiments that surface that is modified with a PEG monolayer comprising terminal hydroxy groups is used to detect the presence of molecules, particles, or cells. In some embodiments, no further modification is performed on the PEG monolayer or brush monolayer comprising terminal hydroxy groups; the modified substrate surface is passivated and nonspecific binding is reduced sufficiently to detect the molecules, particles, or cells of interest, detect the combination of a binding moiety which is not immobilized, bound to its pair in solution, or to modify the surface of vessels, tubing, and apparatus which comes into contact with biological material or is implanted in-vivo. In other embodiments, the surface that is modified with a PEG monolayer comprising terminal hydroxy groups reduces the nonspecific binding of molecules, particles and/or cells is incorporated into tubing, apparatus for bioanalysis, fluid transfer or implantation in-vivo.

In some of the embodiments the PEG monolayer comprising terminal hydroxy groups on the surface of the substrate reduces nonspecific binding by about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% compared to that of a non-passivated surface. In some embodiments the PEG monolayer is a passivated surface that exhibits substantially no nonspecific binding.

In some embodiments of the invention, the PEG monolayer comprising terminal hydroxy groups is the thickness of about 2 ethylenediol repeat units, about 3 ethylenediol repeat units, about 4 ethylenediol repeat units, about 5 ethylenediol repeat units, about 6 ethylenediol repeat units, about 7 ethylenediol repeat units, about 8 ethylenediol repeat units, about 9 ethylenediol repeat units, about 10 ethylenediol repeat units, about 11 ethylenediol repeat units, about 12 ethylenediol repeat units, about 13 ethylenediol repeat units, about 14 ethylenediol repeat units, about 15 ethylenediol repeat units, about 16 ethylenediol repeat units, about 17 ethylenediol repeat units, about 18 ethylenediol repeat units, about 19 ethylenediol repeat units, about 20 ethylenediol repeat units, about 21 ethylenediol repeat units, about 22 ethylenediol repeat units, about 23 ethylenediol repeat units, about 24 ethylenediol repeat units, about 25 ethylenediol repeat units, about 28 ethylenediol repeat units, about 30 ethylenediol repeat units, about 32 ethylenediol repeat units, about 34 ethylenediol repeat units, about 36 ethylenediol repeat units, about 38 ethylenediol repeat units, about 40-ethylenediol repeat units, about 42 ethylenediol repeat units, about 44 ethylenediol repeat units, about 46 ethylenediol repeat units, about 48 ethylenediol repeat units, or about 50 ethylenediol repeat units.

In some embodiments of the invention, the PEG monolayer comprising terminal hydroxy groups is no more than any of: about 400 Ångstroms thick, about 350 Ångstroms thick, about 300 Ångstroms thick, about 290 Ångstroms thick, about 280 Ångstroms thick, about 270 Ångstroms thick, about 260 Ångstroms thick, about 250 Ångstroms thick, about 240 Ångstroms thick, about 230 Ångstroms thick, about 220 Ångstroms thick, about 210 Ångstroms thick, about 200 Ångstroms thick, about 190 Ångstroms thick, about 180 Ångstroms thick, about 170 Ångstroms thick, about 160 Ångstroms thick, about 150 Ångstroms thick, about 140 Ångstroms thick, about 130 Ångstroms thick, about 120 Ångstroms thick, about 110 Ångstroms thick, about 100 Ångstroms thick, about 95 Ångstroms thick, about 90 Ångstroms thick, about 85 Ångstroms thick, about 80 Ångstroms thick, about 75 Ångstroms thick, about 70 Ångstroms thick, about 65 Ångstroms thick, about 60 Ångstroms thick, about 55 Ångstroms thick, about 50 Ångstroms thick, about 45 Ångstroms thick, about 40 Ångstroms thick, about 35 Ångstroms thick, about 30 Ångstroms thick, about 25 Ångstroms thick, about 20 Ångstroms thick, about 19 Ångstroms thick, about 18 Ångstroms thick, about 17 Ångstroms thick, about 16 Ångstroms thick, about 15 Ångstroms thick, about 14 Ångstroms thick, about 13 Ångstroms thick, about 12 Ångstroms thick, about 11 Ångstroms thick, about 10 Ångstroms thick, or about 9 Ångstroms thick.

In some embodiments of the invention, the PEG monolayer comprising terminal hydroxy groups is about 20 to about 400 Ångstroms thick, about 20 to about 380 Ångstroms thick, about 20 to about 360 Ångstroms thick, about 20 to about 340 Ångstroms thick, about 20 to about 320 Ångstroms thick, about 20 to about 300 Ångstroms thick, about 20 to about 280 Ångstroms thick, about 20 to about 260 Ångstroms thick, about 20 to about 240 Ångstroms thick, about 20 to about 220 Ångstroms thick, about 20 to about 200 Ångstroms thick, about 20 to about 180 Ångstroms thick, about 20 to about 160 Ångstroms thick, about 20 to about 140 Ångstroms thick, about 20 to about 120 Ångstroms thick, about 20 to about 100 Ångstroms thick, about 20 to about 80 Ångstroms thick, about 20 to about 60 Ångstroms thick, or about 20 to about 40 Ångstroms thick.

In some embodiments of the invention, the PEG monolayer comprising terminal hydroxy groups is about 30 to about 400 Ångstroms thick, about 30 to about 380 Ångstroms thick, about 30 to about 360 Ångstroms thick, about 30 to about 340 Ångstroms thick, about 30 to about 320 Ångstroms thick, about 30 to about 300 Ångstroms thick, about 30 to about 280 Ångstroms thick, about 30 to about 260 Ångstroms thick, about 30 to about 240 Ångstroms thick, about 30 to about 220 Ångstroms thick, about 30 to about 200 Ångstroms thick, about 30 to about 180 Ångstroms thick, about 30 to about 160 Ångstroms thick, about 30 to about 140 Ångstroms thick, about 30 to about 120 Ångstroms thick, about 30 to about 100 Ångstroms thick, about 30 to about 80 Ångstroms thick, about 30 to about 60 Ångstroms thick, or about 30 to about 40 Ångstroms thick.

In some embodiments of the invention, the PEG monolayer comprising terminal hydroxy groups is about 40 to about 400 Ångstroms thick, about 40 to about 380 Ångstroms thick, about 40 to about 360 Ångstroms thick, about 40 to about 340 Ångstroms thick, about 40 to about 320 Ångstroms thick, about 40 to about 300 Ångstroms thick, about 40 to about 280 Ångstroms thick, about 40 to about 260 Ångstroms thick, about 40 to about 240 Ångstroms thick, about 40 to about 220 Ångstroms thick, about 40 to about 200 Ångstroms thick, about 40 to about 180 Ångstroms thick, about 40 to about 160 Ångstroms thick, about 40 to about 140 Ångstroms thick, about 40 to about 120 Ångstroms thick, about 40 to about 100 Ångstroms thick, about 40 to about 80 Ångstroms thick, or about 40 to about 60 Ångstroms thick.

In some embodiments of the invention, the PEG monolayer comprising terminal hydroxy groups is about 50 to about 400 Ångstroms thick, about 50 to about 380 Ångstroms thick, about 50 to about 360 Ångstroms thick, about 50 to about 340 Ångstroms thick, about 50 to about 320 Ångstroms thick, about 50 to about 300 Ångstroms thick, about 50 to about 280 Ångstroms thick, about 50 to about 260 Ångstroms thick, about 50 to about 240 Ångstroms thick, about 50 to about 220 Ångstroms thick, about 50 to about 200 Ångstroms thick, about 50 to about 180 Ångstroms thick, about 50 to about 160 Ångstroms thick, about 50 to about 140 Ångstroms thick, about 50 to about 120 Ångstroms thick, about 50 to about 100 Ångstroms thick, about 50 to about 80 Ångstroms thick, or about 50 to about 60 Ångstroms thick.

In some embodiments of the invention, the PEG monolayer comprising terminal hydroxy groups is about 60 to about 400 Ångstroms thick, about 60 to about 380 Ångstroms thick, about 60 to about 360 Ångstroms thick, about 60 to about 340 Ångstroms thick, about 60 to about 320 Ångstroms thick, about 60 to about 300 Ångstroms thick, about 60 to about 280 Ångstroms thick, about 60 to about 260 Ångstroms thick, about 60 to about 240 Ångstroms thick, about 60 to about 220 Ångstroms thick, about 60 to about 200 Ångstroms thick, about 60 to about 180 Ångstroms thick, about 60 to about 160 Ångstroms thick, about 60 to about 140 Ångstroms thick, about 60 to about 120 Ångstroms thick, about 60 to about 100 Ångstroms thick, or about 60 to about 80 Ångstroms thick.

In some embodiments of the invention, the PEG monolayer comprising terminal hydroxy groups is about 70 to about 400 Ångstroms thick, about 70 to about 380 Ångstroms thick, about 70 to about 360 Ångstroms thick, about 70 to about 340 Ångstroms thick, about 70 to about 320 Ångstroms thick, about 70 to about 300 Ångstroms thick, about 70 to about 280 Ångstroms thick, about 70 to about 260 Ångstroms thick, about 70 to about 240 Ångstroms thick, about 70 to about 220 Ångstroms thick, about 70 to about 200 Ångstroms thick, about 70 to about 180 Ångstroms thick, about 70 to about 160 Ångstroms thick, about 70 to about 140 Ångstroms thick, about 70 to about 120 Ångstroms thick, about 70 to about 100 Ångstroms thick, or about 70 to about 80 Ångstroms thick.

In some embodiments of the invention, the PEG monolayer comprising terminal hydroxy groups is about 80 to about 400 Ångstroms thick, about 80 to about 380 Ångstroms thick, about 80 to about 360 Ångstroms thick, about 80 to about 340 Ångstroms thick, about 80 to about 320 Ångstroms thick, about 80 to about 300 Ångstroms thick, about 80 to about 280 Ångstroms thick, about 80 to about 260 Ångstroms thick, about 80 to about 240 Ångstroms thick, about 80 to about 220 Ångstroms thick, about 80 to about 200 Ångstroms thick, about 80 to about 180 Ångstroms thick, about 80 to about 160 Ångstroms thick, about 80 to about 140 Ångstroms thick, about 80 to about 120 Ångstroms thick, or about 80 to about 100 Ångstroms thick.

### 2. Surfaces Further Modified by Attachment of a Binding Moiety

2.1. PEG Monolayers with Reactive Groups or Binding Moieties Attached

Substrate surfaces passivated as described above can be used as they are for purposes in which detection of target molecules occurs without any specific binding interaction to a surface. In other uses, binding of a specific binding agent to the surface may be desired. In this case, the PEG layer can be linked to binding agents that will bind particular molecules to the surface. Typically this is accomplished by providing a binding moiety on the PEG, e.g. at the free terminus, and reacting that with the binding partner to couple the binding partner to the surface through a PEG linkage.

2.2. Introduction of Reactive Functionalities at the Free Termini of the PEG Passivated Substrate Surface.

The free termini of the PEG passivated substrate surface are primary hydroxyl groups, which can be reacted with a wide variety of reagents in order to introduce reactive functionalities. The conditions are chosen to convert a portion, not all, of the free hydroxy groups such that there are still enough free hydroxy groups at the termini of the PEG monolayer to reduce the nonspecific binding by non-target molecules, particles or cells.

Some of the reagents which may be used to functionalize the free hydroxy termini include, but are not limited to phosgene, acid chlorides, chloroformates, silanes, methacrylates, disulfides, disilazanes, sulfhydryls, acrylates, carboxylates, activated esters, activated carbonyls such as carbonyldiimazole and other activated species, other active leaving groups such as tosylates, mesylates, or triflates, isonitriles, isocyanates, phosphoamidites, nitrenes, epoxides, hydrosilyl, esters, arenes, azides, amine, nitrile, vinyl groups, alkylphosphonates, or any of the reagents bearing reactive groups at both termini described above.

In some embodiments, the reagent used to functionalize a portion of the free hydroxy termini of the PEG of a passivated substrate surface of the invention is phosgene. In some embodiments, the reagent used to functionalize a portion of the free hydroxy termini of the PEG of a passivated substrate surface of the invention is an acid chloride. In some embodiments, the reagent used to functionalize a portion of the free hydroxy termini of the PEG of a passivated substrate surface of the invention is a chloroformate. In some embodiments, the reagent used to functionalize a portion of the free hydroxy termini of the PEG of a passivated substrate surface of the invention is a silane. In some embodiments, the reagent used to functionalize a portion of the free hydroxy termini of the PEG of a passivated substrate surface of the invention is a methacrylate. In some embodiments, the reagent used to functionalize a portion of the free hydroxy termini of the PEG of a passivated substrate surface of the invention is a disulfide. In some embodiments, the reagent used to functionalize a portion of the free hydroxy termini of the PEG of a passivated substrate surface of the invention is a sulfhydryl. In some embodiments, the reagent used to functionalize a portion of the free hydroxy termini of the PEG of a passivated substrate surface of the invention is a disilazane. In some embodiments, the reagent used to functionalize a portion of the free hydroxy termini of the PEG of a passivated substrate surface of the invention is a acrylate. In some embodiments, the reagent used to functionalize a portion of the free hydroxy termini of the PEG of a passivated substrate surface of the invention is a carboxylate. In some embodiments, the reagent used to functionalize a portion of the free hydroxy termini of the PEG of a passivated substrate surface of the invention is an activated ester. In some embodiments, the reagent used to functionalize a portion of the free hydroxy termini of the PEG of a passivated substrate surface of the invention is an activated carbonyl. In some embodiments, the reagent used to functionalize a portion of the free hydroxy termini of the PEG of a passivated substrate surface of the invention is carbonyldiimazole. In some embodiments, the reagent used to functionalize a portion of the free hydroxy termini of the PEG of a passivated substrate surface of the invention is an active leaving group. In some embodiments, the reagent used to functionalize a portion of the free hydroxy termini of the PEG of a passivated substrate surface of the invention is a tosylate. In some embodiments, the reagent used to functionalize a portion of the free hydroxy termini of the PEG of a passivated substrate surface of the invention is a mesylate. In some embodiments, the reagent used to functionalize a portion of the free hydroxy termini of the PEG of a passivated substrate surface of the invention is a triflate. In some embodiments, the reagent used to functionalize a portion of the free hydroxy termini of the PEG of a passivated substrate surface of the invention is an isonitrile. In some embodiments, the reagent used to functionalize a portion of the free hydroxy termini of the PEG of a passivated substrate surface of the invention is an isocyanate. In some embodiments, the reagent used to functionalize a portion of the free hydroxy termini of the PEG of a passivated substrate surface of the invention is a phosphoroamidite. In some embodiments, the reagent used to functionalize a portion of the free hydroxy termini of the PEG of a passivated substrate surface of the invention is a nitrene. In some embodiments, the reagent used to functionalize a portion of the free hydroxy termini of the PEG of a passivated substrate surface of the invention is an epoxide. In some embodiments, the reagent used to functionalize a portion of the free hydroxy termini of the PEG of a passivated substrate surface of the invention is an ester. In some embodiments, the reagent used to functionalize a portion of the free hydroxy termini of the PEG of a passivated substrate surface of the invention is an arene. In some embodiments, the reagent used to functionalize a portion of the free hydroxy termini of the PEG of a passivated substrate surface of the invention is an azide. In some embodiments, the reagent used to functionalize a portion of the free hydroxy termini of the PEG of a passivated substrate surface of the invention is a nitrile. In some embodiments, the reagent used to functionalize a portion of the free hydroxy termini of the PEG of a passivated substrate surface of the invention is a vinyl moiety. In some embodiments, the reagent used to functionalize a portion of the free hydroxy termini of the PEG of a passivated substrate surface of the invention is an alkylphosphonate.

Reactive functionalities incorporated at the free hydroxy termini of the PEG monolayer may be a number of functionalities including but not limited to epoxide, N-hydroxy succinimide, (NHS), N-hydroxymaleimide (NHM), imidazolecarbonyl, hydrazine, aldehyde, iodoacetyl, cystamine, or dithiothreitol (DTT) groups.

In some embodiments of the invention, the reactive functionality incorporated at the hydroxy termini of the PEG monolayer of the substrate surface is epoxide. In other embodiments of the invention, the reactive functionality incorporated at the hydroxy termini of the PEG monolayer of the substrate surface is N-hydroxy succinimide, (NHS). In some other embodiments of the invention, the reactive functionality incorporated at the hydroxy termini of the PEG monolayer of the substrate surface is N-hydroxymaleimide (NHM). In some embodiments of the invention, the reactive functionality incorporated at the hydroxy termini of the PEG monolayer of the substrate surface is imidazolecarbonyl. In some embodiments of the invention, the reactive functionality incorporated at the hydroxy termini of the PEG monolayer of the substrate surface is hydrazine. In other embodiments of the invention, the reactive functionality incorporated at the hydroxy termini of the PEG monolayer of the substrate surface is an aldehyde. In yet other embodiments of the invention, the reactive functionality incorporated at the hydroxy termini of the PEG monolayer of the substrate surface is iodoacetyl. In some embodiments of the invention, the reactive functionality incorporated at the hydroxy termini of the PEG monolayer of the substrate surface is cystamine. In some embodiments of the invention, the reactive functionality incorporated at the hydroxy termini of the PEG monolayer of the substrate surface is a DTT group.

The reagents used to functionalize a portion of the terminal hydroxyls of the PEG of the passivated substrate surface may themselves be substituted to incorporate a functional group for use in the detection of target molecules, particles, and cells. For example, an activated ester may incorporate an alkyl group which itself comprises an iodoacetyl group, which can be reacted with a moiety, such as, for example, a protein, which will act as a binding moiety to capture its specific binding partner.

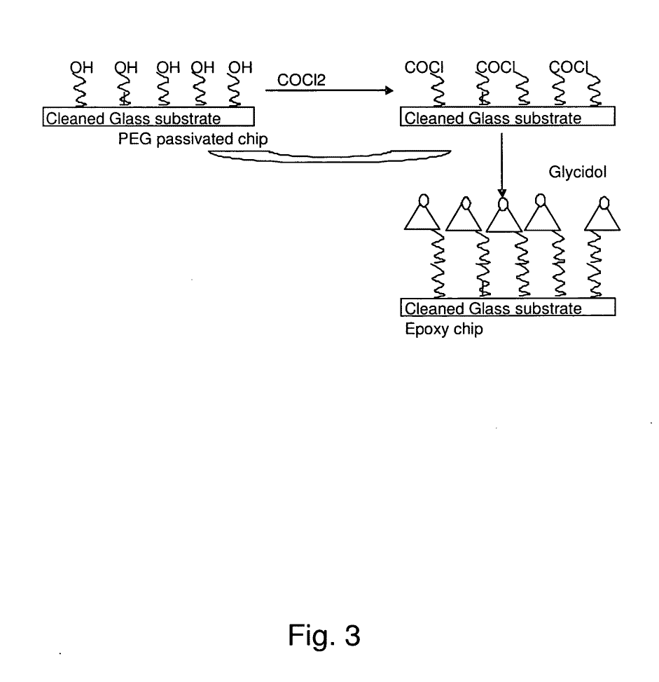

Alternatively the reagents used to functionalize a portion of the terminal hydroxyls of the PEG of the passivated substrate surface may provide moieties which themselves may be further elaborated to provide binding moieties. For example, addition of phosgene to a portion of the terminal hydroxyls of the PEG monolayer provides acid chloride functionalities at the termini, which are then further reacted to introduce binding moieties. A specific example of this is shown in FIG. 3, where epoxide binding moieties are introduced at a portion of the termini of a PEG 5mer brush monolayer on a glass substrate, which can react with a protein or antibody which then acts as a binding moiety to capture its specific biological binding partner. Another example is where a NHS moiety is introduced at the termini of a PEG brush monolayer which is further reacted with a protein or antibody which can then act as a binding moiety.

2.3. Binding Moieties

Binding moieties can be a biomolecule, an organic molecule, a synthetic molecule, a particle, or a cell. Examples of a biomolecule useful in this invention as a binding moiety include but are not limited to an antibody, a Fab, a Fc, a single chain variable fragment (scFv), nucleic acid, protein, lectin, phosphatidyl glycol, antigen, receptor, receptor ligand, enzyme, lipoprotein, lipid, hapten or an aptamer. Examples of organic molecules useful as binding moieties include but are not limited to avidin, biotin, hapten, organic molecules, drug molecules, small organometallic compounds, or a carbohydrate. Examples of particles capable of being used as a binding moiety in the articles and methods of the invention include, but are not limited to cell organelles, viruses, viral particles, cell fragments, or membranes. Examples of cells capable of being used as a binding moiety in the articles or methods of the invention include but are not limited to abnormal, normal or rare cells of mammals, viruses, or bacteria.

In some of the embodiments of the invention, a binding moiety is an antibody. In some of the embodiments of the invention, a binding moiety is a Fab. In some of the embodiments of the invention, a binding moiety is a Fc. In some of the embodiments of the invention, a binding moiety is a nucleic acid. In some of the embodiments of the invention, a binding moiety is a protein. In some of the embodiments of the invention, a binding moiety is a post-translationally modified protein including but not limited to protein fragments, glyco-proteins, phospho-proteins, peptides and the like. In some of the embodiments of the invention, a binding moiety is a to protein fragments. In some of the embodiments of the invention, a binding moiety is a glycoprotein. In some of the embodiments of the invention, a binding moiety is a phospho-protein, In some of the embodiments of the invention, a binding moiety is a peptide. In some of the embodiments of the invention, a binding moiety is a lectin. In some of the embodiments of the invention, a binding moiety is a phosphatidyl glycol. In some of the embodiments of the invention, a binding moiety is an antigen. In some of the embodiments of the invention, a binding moiety is a receptor. In some of the embodiments of the invention, a binding moiety is a receptor ligand. In some of the embodiments of the invention, a binding moiety is an enzyme. In some of the embodiments of the invention, a binding moiety is a lipoprotein. In some of the embodiments of the invention, a binding moiety is a lipid. In some of the embodiments of the invention, a binding moiety is a hapten. In some of the embodiments of the invention, a binding moiety is an aptamer. In some of the embodiments of the invention, a binding partner is avidin. In some of the embodiments of the invention, a binding moiety is biotin. In some of the embodiments of the invention, a binding moiety is a synthetic organic molecule. In some of the embodiments of the invention, a binding moiety is a natural product molecule. In some of the embodiments of the invention, a binding moiety is a drug molecule. In some of the embodiments of the invention, a binding moiety is an organometallic compound. In some of the embodiments of the invention, a binding moiety is a carbohydrate. In some of the embodiments of the invention, a binding moiety is a cell organelle. In some of the embodiments of the invention, a binding moiety is a virus. In some of the embodiments of the invention, a binding moiety is a viral particle. In some of the embodiments of the invention, a binding moiety is a cell fragment. In some of the embodiments of the invention, a binding moiety is a membrane. In some of the embodiments of the invention, a binding moiety is bacteria. In some of the embodiments of the invention, a binding moiety is a normal mammalian cell. In some of the embodiments of the invention, a binding moiety is a normal virus. In some of the embodiments of the invention, a binding moiety is a normal bacterium. In some of the embodiments of the invention, a binding moiety is an abnormal mammalian cell. In some of the embodiments of the invention, a binding moiety is an abnormal virus. In some of the embodiments of the invention, a binding moiety is an abnormal bacterium. In some of the embodiments of the invention, a binding moiety is a rare mammalian cell. In some of the embodiments of the invention, a binding moiety is a rare virus. In some of the embodiments of the invention, a binding moiety is a rare bacterium. In some of the embodiments of the invention, a binding moiety is a Molecular Imprint Polymer (MIP). In some of the embodiments of the invention, a binding moiety is a single chain antibody fragment (SFv).

Binding moieties have specific binding partners. The binding moieties used in the compositions of the invention are selected for use with a binding partner. Binding partners can be a biomolecule, an organic molecule, a synthetic molecule, a particle, or a cell. Examples of a biomolecule useful in this invention include but are not limited to an antibody, a Fab, a Fc, nucleic acid, protein, lectin, phosphatidyl glycol, antigen, receptor, receptor ligand, enzyme, lipoprotein, lipid, hapten or an aptamer. Examples of useful organic molecules include but are not limited to avidin, biotin, hapten, organic molecules, drug molecules, small organometallic compounds, or a carbohydrate. Examples of particles capable of being used as a binding partner in the articles of the invention include, but are not limited to cell organelle, viruses, cell fragments, or membranes. Examples of cells capable of being used as a binding partner in the articles of the invention include but are not limited to abnormal, normal or rare mammalian cells, viruses, or bacteria.

In some of the embodiments of the invention, a binding partner is an antibody. In some of the embodiments of the invention, a binding partner is a Fab. In some of the embodiments of the invention, a binding partner is a Fc. In some of the embodiments of the invention, a binding partner is a nucleic acid. In some of the embodiments of the invention, a binding partner is a protein. In some of the embodiments of the invention, a binding partner is a lectin. In some of the embodiments of the invention, a binding partner is a phosphatidyl glycol. In some of the embodiments of the invention, a binding partner is an antigen. In some of the embodiments of the invention, a binding partner is a receptor. In some of the embodiments of the invention, a binding partner is a receptor ligand. In some of the embodiments of the invention, a binding partner is an enzyme. In some of the embodiments of the invention, a binding partner is a lipoprotein. In some of the embodiments of the invention, a binding partner is a lipid. In some of the embodiments of the invention, a binding partner is a hapten. In some of the embodiments of the invention, a binding partner is an aptamer. In some of the embodiments of the invention, a binding partner is avidin. In some of the embodiments of the invention, a binding partner is biotin. In some of the embodiments of the invention, a binding partner is a synthetic organic molecule. In some of the embodiments of the invention, a binding partner is a natural product molecule. In some of the embodiments of the invention, a binding partner is a drug molecule. In some of the embodiments of the invention, a binding partner is an organometallic compound. In some of the embodiments of the invention, a binding partner is a carbohydrate. In some of the embodiments of the invention, a binding partner is a cell organelle. In some of the embodiments of the invention, a binding partner is a virus. In some of the embodiments of the invention, a binding partner is a viral particle. In some of the embodiments of the invention, a binding partner is a cell fragment. In some of the embodiments of the invention, a binding partner is a membrane. In some of the embodiments of the invention, a binding partner is bacteria. In some of the embodiments of the invention, a binding partner is a normal mammalian cell. In some of the embodiments of the invention, a binding partner is a normal virus. In some of the embodiments of the invention, a binding partner is a normal bacterium. In some of the embodiments of the invention, a binding partner is an abnormal mammalian cell. In some of the embodiments of the invention, a binding partner is an abnormal virus. In some of the embodiments of the invention, a binding partner is an abnormal bacterium. In some of the embodiments of the invention, a binding partner is a rare mammalian cell. In some of the embodiments of the invention, a binding partner is a rare virus. In some of the embodiments of the invention, a binding partner is a rare bacterium. In some of the embodiments of the invention, a binding partner is a MIP. In some of the embodiments of the invention, a binding partner is a SFv.

### 3. Articles Passivated with PEG Monolayer

This invention contemplates a variety of articles having surfaces coated at least in part with the PEG monolayers of this invention.

3.1. Articles with Channels Passivated with PEG Monolayer Comprising Terminal Hydroxy Groups.

This invention further provides articles or devices that comprise a substrate having a channel in which the channel surface is passivated with a PEG monolayer according to this invention. The substrate can be any useful substrate including those already described herein. These articles can take any form in which it is useful to avoid non-specific binding of biomolecules to a surface, including analytical instrumentation and medical devices meant for implantation.

In some embodiments of the invention, the articles comprise at least one channel in a substrate. In some embodiments the channel is a groove in the substrate. In some of the embodiments of the invention, the channel is formed by sandwiching one piece of substrate which has a groove in the substrate to a second piece of substrate which has no groove formed in it. In some embodiments, when two pieces of substrate are sandwiched together, the two facing surfaces of the two pieces have been passivated with a PEG monolayer according to this invention. Sandwiches can be created using any methods compatible with the substrate used. For example, adhesives for any variety of substrate materials are known in the art and can be used for glass or plastic. The pieces can be held tougher with chemical bonding. The pieces can be physically held together, for example by a clamp. In the case of glass, glass pieces can be heated to just below the transition temperature and bonded together. Plastics can also be formed by injection molding, eradicating the need to join two halves together.

In some embodiments the channel is drilled out of the substrate. In some embodiments, the substrate with a channel in it has been formed by injection molding or by microinjection molding to eliminate the requirement for sandwiching two pieces of substrates together.

In some embodiments there is more than one channel in the substrate. In other embodiments there are more than two channels in the substrate.

In some embodiments, the channel is a microfluidic channel. Microfluidic channels generally have a cross sectional area of less than 1 mm². In other embodiments, the channel is semicircular. In yet other embodiments, the channel is fabricated with a rectangular or square cross sectional area. In other embodiments, the channel has cross sectional area of about of about 0.01 mm², about 0.02 mm², about 0.03 mm², about 0.04 mm², about 0.05 mm², 0.06 mm², about 0.07 mm², about 0.08 mm², about 0.09 mm², about 0.1 mm², about 0.2 mm², about 0.3 mm², about 0.4 mm², about 0.5 mm², about 0.6 mm², about 0.7 mm², about 0.8 mm², about 0.9 mm², or about 1.0 mm².

In other embodiments the channel has a diameter no greater than any of: about 1.0×10⁴ μm, about 9×10³ μm, about 8×10³ μm, about 7×10³ μm, about 6×10³ μm, about 5×10³ μm, about 4×10³ μm, about 3×10³ μm, about 2×10³ μm, about 1×10³ μm, about 9×10² μm, about 8×10² μm, about 7×10² μm, about 6×10² μm, about 5×10² μm, about 4×10² μm, about 3×10² μm, about 2×10² μm, about 1×10² μm about 9×10 μm, about 8×10 μm, about 7×10 μm, about 6×10 μm, about 5×10 μm, about 4×10 μm, about 3×10 μm, about 2×10 μm, about 1×1 μm, about 9 μm, about 8 μm, about 7 μm, about 6 μm, about 5 μm, about 4 μm, about 3 μm, about 2 μm, about 1 μm, about 0.9 μm, about 0.8 μm, about 0.7 μm, about 0.6 μm, about 0.5 μm, about 0.4 μm, about 0.3 μm, about 0.2 μm, or about 0.1 μm.

In other embodiments the channel has a diameter no greater than 500 μm.

In some embodiments of the invention, the article comprises two pieces wherein each piece comprises a surface with a PEG monolayer. In some embodiments, the terminal hydroxyl groups of at least one of the surfaces of the two pieces which comprise a PEG monolayer is coupled to a reactive functionality. The two pieces are sandwiched together with the two surfaces comprising the PEG monolayer facing each other and the pieces are bonded to each other when the reactive functionality on one of the two surfaces comprising a PEG monolayer reacts with the PEG monolayer on the surface comprising the PEG monolayer of the second piece, which faces it. In some embodiments, both surfaces have reactive functionalities coupled to the PEG monolayer. Shorter PEGS, e.g., PEG 200, are useful for this purpose. In some embodiments the reactive functionality is epoxy, methacrylate or acrylate. In another embodiment, glass coated with PEF can be reacted with COCl₂ to produce a reactive group that can be bonded with PEG on an opposing glass layer. The pieces can be bonded using pressure and temperature. In some embodiments the reaction occurs at room temperature. This method can be used with any substrate to which PEG can be attached, including glass and plastic. Methods of derivatizing glass with PEG have been described herein. Plastics can be treated to plasma oxidation or corona discharge to introduce groups to which PEG can be bound.

3.2. Other Articles Passivated with PEG Monolayer Comprising Terminal Hydroxy Groups

Other types of bioanalytical chips/assay surfaces or reaction modules may be passivated with the brush monolayer of PEG comprising terminal hydroxy groups, including arrays of proteins, nucleic acids or other biomolecules. The format may be capillary tubes, pipette tips, microwells, microliter plates, microtubes, arrays with addressable sites, waveguides, compact discs, or chips with continuous lanes or contiguous surfaces. The fluidic conduits which conduct a sample to the detection zone, fluidic or biologics handling apparatus such as microtubes, microcentrifuge tubes, micropipettes, disposable pipette tips, and other dispensing means, and any of the components of bioanalytical apparatus that contacts such biological molecules, organic and synthetic molecules, particles and cells.

For uses outside of bioanalytical detection, almost any article may be passivated with a brush monolayer of PEG comprising terminal hydroxy groups of the invention. Equipment contacting biological materials of many types, including but not limited to blood, plasma, cell cultures, tissue, or any biological material or fluid obtained from a living creature may be passivated with a brush monolayer of PEG comprising terminal hydroxy groups. One embodiment of such apparatus is, for example, tubing for intravenous supply or withdrawal of plasma to or from a mammal. The modified surfaces may also be used to coat medical devices such as stents, valves, sutures, surgical staples, or prostheses, such as joint replacement inserts which are implanted in a mammal, to prevent inappropriate cell attachment.

These materials may also be used for industrial applications such as, for example, tubing, containers, or transfer apparatus for food processing. Additionally, the passivated monolayers of PEG comprising terminal hydroxy groups of the invention may be used for specialty manufacturing apparatus when handling, containing, or transferring materials, particularly hydrophobic materials which may create drag, static, or unacceptable viscosity when interacting with untreated surfaces.

### 4. Methods of Use

The articles of the invention are used to detect molecules, particles, or cells. Molecular interaction can be detected by any suitable method. Detection paradigms include optical methods, electrochemical methods (voltammetry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

In some embodiments, the article is a substrate which is a chip which comprises at least one channel. In some embodiments, the chip comprises at least two channels. In some embodiments, the chip comprises a plurality of channels. In some embodiments the at least one channel is a microfluidic channel. In some embodiments the at least one channel has a cross sectional area large enough permit passage of cells. In some embodiments the at least one channel has a cross sectional area large enough to permit passage of cell fragments, membranes or cell organelles, but is not large enough to permit passage of cells.

In some embodiments, the at least one channel has a wall and the wall of the channel is passivated by covalently attaching a monolayer of PEG, according to the methods of the invention. In some embodiments, the monolayer of PEG which passivates the wall of the at least one channel, has no reactive groups coupled to the terminal hydroxyl groups. In some embodiments, the monolayer of PEG which passivates the wall of the at least one channel, is coupled covalently to a binding moiety. The binding moiety is attached to the wall of the at least one channel via covalent bonds through the PEG acting as a linker to the wall and/or surface of the substrate.

In some embodiments, wherein the at least one channel comprises a binding moiety attached to the wall via covalent bonds to a PEG monolayer which passivates the surface of the wall, fluid which comprises a binding partner to the binding moiety is flowed through the at least one channel. The binding partner binds to the binding moiety which is attached to the wall of the at least one channel and is captured. In some embodiments, excess binding moieties are blocked from further reaction after the binding partner has been captured and before detection of the binding partner. The excess binding moieties are blocked by flowing fluid though the at least one channel which comprises a blocking reagent. For example, when the binding moieties are epoxy, excess binding moieties are blocked, for example, by ethanolamine.

The presence of the binding partner is detected after capture. Detection may be made by any suitable means. Depending on the substrate utilized, fluorescence detection, interferometry, and in particular back scattering interferometry, chemical sensing interferometry, surface plasmon resonance (SPR), grating couple waveguide interferometry or refractive index detection, biolayer interferometry, acoustic detection means, atomic force microscopy vibrating cantelever means, piezoelectric detection, voltammetric detection, amperometric detection, ellipsometric detection, calorimetry, isothermal titration calorimetry, chemiluminescence, radiometric detection, micro Fourier Transform Infrared Spectrometry (FTIR), quartz crystal microbalance (QCM), fluorescence quenching, affinity chromatography, or mass spectrometry may be utilized. In some embodiments of the invention, back-scattering interferometry (BSI) detection of molecules is performed. BSI detection is amenable to high throughput assay methods, as the molecules, particles or cells do not require labeling with other reagents, such as fluorescent tags, thus requiring less processing of individual samples. The presence of the mass of the immobilized target or a signal due to a binding pair in solution, in embodiments where no binding moiety is immobilized, is detected directly as a function of BSI signal and is robust under laser interrogation. The resulting signal is not susceptible to the photobleaching and loss of precision under long or repeated laser exposure of fluorescently labeled targets. BSI detection is a sensitive method of detection. Femtomolar levels of numbers of molecules can be detected and low picomolar (10⁻¹²) concentrations of target molecules can be detected.

Analytes can be detected on the passivated articles of this invention by BSI. The methods involve, for example, providing a chip having a channel formed therein for reception of a sample to be analyzed; disposing a sample to be analyzed in said channel; directing an incident beam of monochromatic, coherent light onto said channel; detecting an interference fringe pattern that is generated by said channel and said sample when said beam or beams of radiation is incident thereon and results in the emission of a new beam or beams whose phase is the result of a series of reflection and refractive events within the probed channel reflected/refracted thereby creating an interference fringe pattern; and determining from said interference fringe pattern, whether said emitted beams of radiation creates a new fringe pattern which is measurably different when compared to a reference fringe pattern. The method can involve capturing the analyte on a binding reagent immobilized to the surface of such a channel. Alternatively, the process can involve introducing two free-solution, unlabeled binding partners into the channel and detecting the interaction by BSI.

Many different kinds of pairs of binding partners are detected in this method. This includes but is not limited to the pair of binding partners which has a binding moiety which is an antibody which is immobilized on the PEG monolayer and a hapten which is the binding partner in the fluid being flowed through the at least one channel. Alternatively, the hapten is the binding moiety immobilized on the PEG monolayer, and the antibody is in the fluid which is flowing through the at least one channel. Another binding pair is a receptor protein which is the binding moiety immobilized on the PEG monolayer and the binding partner is a receptor ligand or a small molecule which is comprised in the fluid flowing through the at least one channel. Cells, cell fragments or cell organelles may be the binding partner flowing in the fluid through the at least one channel, and is captured by a binding moiety which is an immobilized protein receptor with specific affinity for a surface protein, or a binding moiety which is an immobilized antibody with specific affinity for a surface antigen on the cell, cell fragment or organelle. Some examples, include but are not limited to protein A-IgG, Protein G-IgG, IL-8-anti-IL-8, and CRP-anti-CRP. Many other pairs are known in the art and can be selected without undue experimentation. Some of the possible binding moieties and binding partners which form binding pairs are discussed elsewhere in this disclosure. Specific capture of targets and monitoring of inter-molecular affinity are performed by the use of the methods herein. Quantification of a target analyte is performed by the methods herein. One example is the use of a capture molecule to specifically capture and bind its binding partner, and to quantify the resultant capture, This functions similarly to an ELISA but does not require enzymatic amplification or the use of fluorescent labeling which adds expense and time to the analysis as well as the possibility of altering the binding interaction, with the insertion of the label.

### 5. Back Scattering Interferometry

A back-scattering interferometer typically comprises an optical assembly and electronics to analyze an optical signal. The optical assembly can be mounted on an optical bench. Back-scattering interferometers are well known in the art. They are described, for example, in U.S. Pat. Nos. 5,325,170, 6,381,025; 6,809,828 and 7,130,060; International applications WO 2004/023115, WO 2006/047408 and WO 2009/039466; U.S. patent publication U.S. 2006-0012800 and U.S. patent publication 2009-0185190.

The optical assembly comprises the following elements: First, a fluidic container having compartment for holding a sample. A portion of the container in which the sample is contained functions as a sensing area or detection zone. Second, the optical assembly comprises a coherent light source positioned to direct a beam toward the sensing area, wherein the path of the beam defines an optical train and generates a back-scattering light pattern, also called an interference fringe pattern. Third, the optical assembly comprises a photodetector configured to detect the back-scattering light pattern. Typically, the instrument also will comprise a computer that converts the fringe pattern into a measure or indicator of refractive index. Optionally, the instrument comprises a temperature regulator that can maintain a stable temperature at least within the fluid during periods of measurement.

Several factors influence the generation of an interference pattern: Reflection, refraction and retardation (of the light beam). The coherent light beam should be large enough so that it passes across a non-flat surface from the container into the liquid. Accordingly, the compartment should comprise a curve or an edge (e.g., a corner) through which the light passes in order to generate a useful interference pattern.

5.1. The Container

The container used in this invention is adapted for use in back scattering interferometry. The container is adapted to generate a backscatter fringe pattern when filled with liquid and interrogated with a focused or unfocused coherent light source, such as a laser beam. Factors that influence the ability to create such a pattern include the relative refractive indices of the substrate that forms the container and the liquid within, as well as the shape of compartment in which the liquid is contained and the light source strikes.

The container can take the shape of a chip (e.g., a microchip). As in known in the art, chips can accommodate a plurality of channels or other features due to having one very thin dimension compared with their other dimensions. The container also can take the shape of a tube, such as a microcapillary tube.

5.1.1. Container Material

The container should be made of a material that has a different (e.g., higher) refractive index than the sample inside. The container can be formed of any suitable optically transmissive material, such as glass, quartz, borosilicate, silica (e.g., fused silica) or a polymeric material, e.g., a plastic such polystyrene, polysulfone, polyetherimide, polyethersulfone, polysiloxane, polyester, polycarbonate, polyether, polyacrylate, polymethacrylate, cellulose, nitrocellulose, a perfluorinated polymer, polyurethane, polyethylene, polyamide, polyolefin, polypropylene or nylon.

5.1.2. Compartment Shape and Size

The container will have an internal compartment that can hold the sample. Typically, the compartment will take the shape of a bore. The bore may have a curved cross section that is, for example, circular, substantially circular, hemicircular, rectangular or elliptical. Backscatter fringe patterns are easily produced with when the substrate includes a compartment having curved or angular walls through which the light passes to reach the sample.

In certain embodiments, the compartment takes a long, thin shape, such as a channel, column, cylinder or tube.

The container also is adapted to receive a liquid sample. In certain embodiments, the container is adapted to function as the collection unit of the sample from its primary source, e.g., a subject organism. For example, the container can comprise a channel or tube that opens at two ends of the container. For example, the container can be a capillary tube or a hematocrit tube, or a chip comprising a channel that opens at different sides of the chip.

The container can take the shape of a capillary tube or microhemotcrit tube. The tube can be, for example, approximately 75 mm long, with fire-polished ends that can easily be sealed if desired. Tube can be coded with a red band to designate heparin coating. It can contain at least 2 U.S.P. units of cation-free ammonium heparin. It can have an I.D. is 1.1 to 1.2 mm with a wall of 0.2 mm±0.02. The volume of the compartment can be between 100 nanoliters and 1000 microliters (10 milliliters), between 1 microliter and 1 milliliter, between 10 microliters and 1 milliliter or between 50 microliters and 250 microliters. Furthermore the tube can have dimensions as follows: Outside diameter 0.75 to 2.0 mm, inside diameter from 0.05 to 1.5 mm.

In some embodiments, the channel is a microfluidic channel. Microfluidic channels generally have a cross sectional area of less than 1 mm². In other embodiments, the channel has cross sectional area of about of about 0.01 mm², about 0.02 mm², about 0.03 mm², about 0.04 mm², about 0.05 mm², 0.06 mm², about 0.07 mm², about 0.08 mm², about 0.09 mm², about 0.1 mm², about 0.2 mm², about 0.3 mm², about 0.4 mm², about 0.5 mm², about 0.6 mm², about 0.7 mm², about 0.8 mm², about 0.9 mm², or about 1.0 mm².

In other embodiments the channel has a diameter no greater than any of: about 1.0×104 μm, about 9×103 μm, about 8×103 μm, about 7×103 μm, about 6×103 μm, about 5×103 μm, about 4×103 μm, about 3×103 μm, about 2×103 μm, about 1×103 μm, about 9×102 μm, about 8×102 μm, about 7×102 μm, about 6×102 μm, about 5×102 μm, about 4×102 μm, about 3×102 μm, about 2×102 μm, about 1×102 μm about 9×10 μm, about 8×10 μm, about 7×10 μm, about 6×10 μm, about 5×10 μm, about 4×10 μm, about 3×10 μm, about 2×10 μm, about 1×1 μm, about 9 μm, about 8 μm, about 7 μm, about 6 μm, about 5 μm, about 4 μm, about 3 μm, about 2 μm, about 1 μm, about 0.9 μm, about 0.8 μm, about 0.7 μm, about 0.6 μm, about 0.5 μm, about 0.4 μm, about 0.3 μm, about 0.2 μm, or about 0.1 μm. In other embodiments the channel has a diameter no greater than 500 μm.

In certain embodiments the analyte is detected as a result of its binding to a binding agent. In this case, the binding agent for an analyte in a sample that one is testing for can be immobilized on the wall of the compartment (heterogeneous assay) or allowed to remain free in solution after the sample is added (homogeneous assay). Binding partners include, for example, antibodies and antibody-like molecules, receptors, nucleic acids (e.g., oligonucleotides). In another embodiment, the agent can be an enzyme or enzyme complex (mixture) which catalyzes an enzymatic reaction which can degrade sample components such as cells, cell fragments, and/or biomolecules. In another embodiment the agent could be an enzyme or enzyme complex (mixture) which catalyzes the creation of new biomolecules arising from the fusion of biomolecular species (such as a ligase) or replication—amplification of biomolecular species, as is the case in polymerase chain reactions.

Moreover, the surfaces of the sample container could be coated with a material to minimize unwanted interactions with the walls of the container. Such surfaces would include polymeric coatings, such as dextran, TEFLON® (polytetrafluoroethylene), polyethylene glycol, etc. Furthermore, the surfaces of the container could be coated with biospecific reagents for selective capture of target analytes or selective enzymatic modification of target analytes as described above.

5.2. Container Mounting/Temperature Regulation

The device of this invention typically comprises a mounting adapted to receive the container and position it for interrogation by the coherent light source. The mounting can be removable from the frame of the device. The mounting can be attached to an optical bench that comprises other components of the optical system. The mounting can comprise a fastener to fasten the container to the mounting. If the container is a tube, the mounting can comprise, for example, a clip or set of clips, a surface with an indentation adapted to receive the tube, in which it can rest, an adhesive material, or a holder in which the container is inserted and held, e.g., a cylinder in which a tube is slid within and retained, a flat mounting stage on which a chip is locked into position. In certain embodiments the mount is in thermal contact with a temperature control assembly such as a Peltier device to insure homogeneous control of temperature as required to perform high sensitivity BSI measurements (+/−1-5 millidegree C.). See, for example, U.S. patent application Ser. No. 12/331,354, filed Dec. 9, 2008.

A container of the invention can be adapted and configured to fit snugly within a holder. The container can be held in place by a positioner, such as a metal plate with tightening screws. The container can be manually inserted into the holder or cartridge. In an embodiment, the container is disposable while the holder can be used for numerous different chips with a device of the invention. A holder retention mechanism can be used to firmly hold the chip in the holder along the axis of the mechanism. The container and/or the thermal subsystem can be affixed to a translation stage that allows adjustment of the chip relative to the laser beam. For example, the container can be tilted slightly (for example, approximately 7°) so that the backscattered light from the sensing area of the container can be directed onto the photodetector.

In experiments that involve comparing the interference pattern between two samples (e.g., a test and control sample), the samples can be measured simultaneously or in sequence. In simultaneous measurements the two samples can be loaded onto the interferometer and a beam splitter can split the laser beam and direct it to each of the two samples. Alternatively, the beam can be made wide enough so that a single beam covers both fluid compartments. In one embodiment, the first and second samples are comprised in different containers, e.g., tubes, and one tube is tilted or rotated, e.g., 3° to 7° with respect to the other tube. This results in the interference signal from each container being directed to different parts of the detector so that they are distinguishable.

In another embodiment, the first and second samples are located within a single tube, where the first sample represents a region of the sample container that contains a selectively deposited binding molecule for extraction and subsequent analysis of a target of interest, and where the second or reference sample represents a region of the sample container that is free of binding molecule, or moreover is coated with a specific passivating agent to minimizing unwanted non-specific binding of the target of interest.

Sample can be introduced into the container by any method known. For example, the sample can be introduced manually using a syringe, e.g., manual pipetter. Also, sample can be introduced into the container using a fluidics robot, such as any commercially available robot, e.g., from Beckman or Tecan.

5.3. Coherent Light Source

Examples of coherent light sources for use with the invention include, but are not limited to, a laser, for example a He/Ne laser, a vertical cavity surface emitting laser (VCSEL) laser, and a diode laser. The coherent light may be coupled to the site of measurement by known wave-guiding or diffractive optical techniques or may be conventionally directed to the measurement site by free space transmission. The coherent light is preferably a low power (for example, 3-15 mW) laser (for example, a He/Ne laser). As with any interferometric technique for chemical analysis, the devices and methods of the invention benefit from many of advantages lasers provide, including high spatial coherence, monochromaticity, and high photon flux. The beam can be directed directly to a sensing area on the fluidic chamber or to a mirror that is angled with respect to the plane of propagation of the laser beam, wherein the mirror can redirect the light onto the sensing area. In another embodiment, the coherent light is preferably generated by a solid state laser source such as a light emitting diode or vertical cavity surface emitting laser (VCSEL), for which requisite beam characteristics of monochromaticity and beam coherence is achieved. In an embodiment, the coherent light source generates an easy to align collimated laser beam that is incident on a sensing area of the container for generating the backscattered light.

5.4. Detector

A photodetector can be configured and incorporated into a device of the invention to detect a fringe pattern produced by back-scattered light from a sensing area on a container. The pattern is based on the contents and/or composition of the sample. In an embodiment, qualitative and quantitative measurements are performed by forming molecular complexes, such as antibody-antigen or drug target-drug candidate. In an embodiment, the photodetector detects a qualitative or quantitative value of an analyte in a liquid sample, for example, the amount of a specific antigen in a blood sample or host antibody titer towards a given antigen.

The photodetector can be one of any number of image sensing devices, including a bi-cell position sensor, a linear or area array CCD or CMOS camera and laser beam analyzer assembly, a slit-photodetector assembly, an avalanche photodiode, or any other suitable photodetection device. The backscattered light comprises interference fringe patterns that result from the reflective, refractive, and retardation interaction of the incident laser beam with the walls of the sensing area and the sample. These fringe patterns include a plurality of light bands whose positions shift as the refractive index of the sample is varied, for example, through compositional changes. For example, a sample in which two components bind to each other can have a different refractive index than a sample in which the two components do not bind. In an embodiment, the photodetector detects the backscattered light and converts it into one or more intensity signals that vary as the positions of the light bands in the fringe patterns shift. For fringe profiling, the photodetector can be mounted above the chip at an approximately 45° angle thereto. Fringe profiling can also be accomplished by detecting the direct backscatter. In an embodiment, the fringes can be profiled in direct backscatter configuration and direct them onto the camera which is at 90° from the beam, in this way, the packaged device can remain small while maximizing the resolution for measuring a positional shift, for example, the effect of angular displacement.

5.5. Detection

The photodetector can detect the backscattered light fringe pattern and, in combination with computer algorithms, convert it into signals that can be used to determine a parameter of refractive index (RI), or an RI related characteristic property, of the sample. For example, the RI of a sample with a certain concentration of analyte in the sample can be slightly different than the RI of a sample where the analyte is present in the sample in a different concentration. A signal analyzer, such as a computer or an electrical circuit, can be employed to analyze the photodetector signals and determine the characteristic property of the sample. Positional shifts in the light bands relative to a baseline or a reference value can then be detected by a photodetector and computed using a processor, such as a PC. The device can provide a signal (for example, positional shifts in the light bands) that is proportional to abundance of the analyte. Preferably, the signal analyzer includes the programming or circuitry necessary to determine from the positional shift of the formed fringes, the RI or other characteristic properties of the sample to be determined, such as temperature or flow rate, for example. The parameter of refractive index can be, for example, the position of the bands on some scale of location. This position can be displayed as a number or as coordinate on a graph. For example, the coordinate on the Y axis can change over time on the X axis. The parameter can be quantitatively related to sample refractive index.

The signal analyzer can be a computer which, optionally, controls other aspects of the system. The computer functions to perform the calculations necessary to detect the fringe movement and output the data on the user interface. Moreover, the computer can function to store and retrieve method files which automate the performance of an assay or analysis, provides data analysis tools to determine binding profiles, qualitative measurements, and quantitative measurements, as well as providing a means to calibrate the system for total gain and output based upon a reference sample.

The photodetector can be a camera, such as a CCD camera. The camera captures the image of the fringe pattern. A CCD camera can typically collect from one to sixty images per second. The image can be projected on a monitor for visual analysis. For example, the monitor can be calibrated and/or the operator can visually detect changes in the fringe pattern over time. Alternatively, the image can be subjected to a variety of mathematical algorithms to analyze the fringe pattern. Examples of algorithms used to analyze fringe pattern are Fourier transforms, Gaussian fit with or without hamming window and sinusoidal correction.

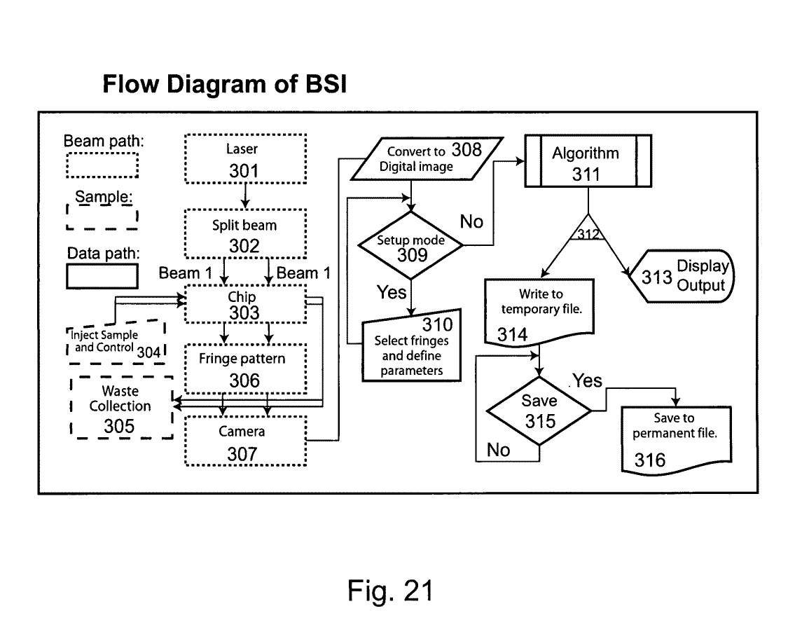

FIG. 21 depicts a flow diagram of a BSI system. A laser 301 produces a beam that passes through a beam splitter 302 to create two beams. A beam splitter is optional but useful for comparing first and second samples. These two beams impinge onto chip 303. The two-channel chip allows for the injection of samples and controls 304. The liquid that is injected passes through chip 303 and then is collected as waste 305. The interaction of the beams and the channels creates fringe patterns 306. These two fringe patterns 306 are directed onto camera 307. The data acquired from camera 307 is converted into digital image 308. Initially, the program is started in setup mode 309, which allows the user to select the fringes to be analyzed and define the parameters of analysis 310. Once setup mode 309 is turned off, digital image 308 is passed to an algorithm 311 that calculates shifts in fringe patterns 306. This output is split 312 to real time output display 313 and is also written to temporary file 314. At any time the user can save the data 315, which then writes the data to permanent file 316.

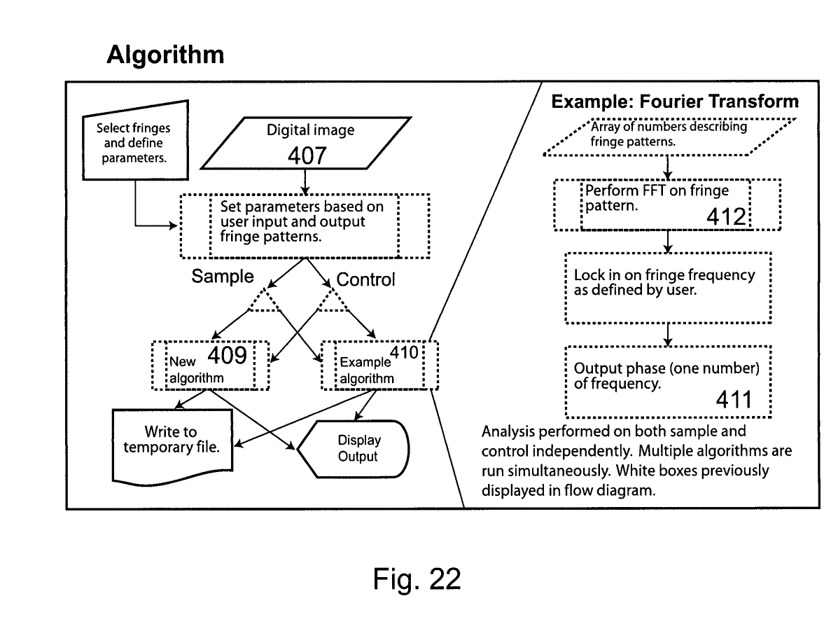

FIG. 22 shows a diagram of the digital image process 401 with the Fourier transform algorithm as an example. The Fourier transform transforms a digital image into functions 409 and 410 that describe the image. Phase changes for the predominant spatial frequency 411 in the Fourier transform over time can indicate shifts in fringe pattern 412.

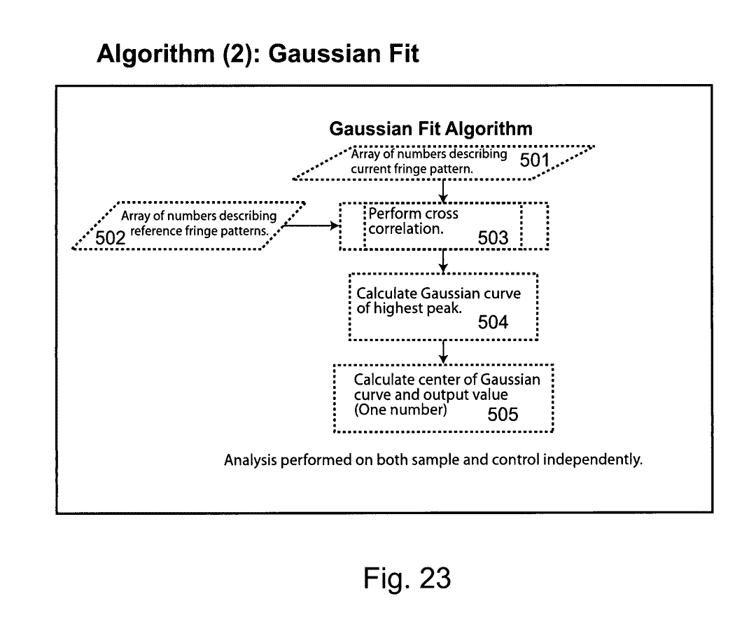

FIG. 23 shows the Gaussian fit analysis. A cross correlation 503 is performed on reference fringe pattern 502 and new pattern 501. A Gaussian fit is calculated 504 from the highest peak of the cross correlation. The calculated center of the Gaussian fit 505 is used to measure the pixel shift, which allows for sub-pixel shift detection.

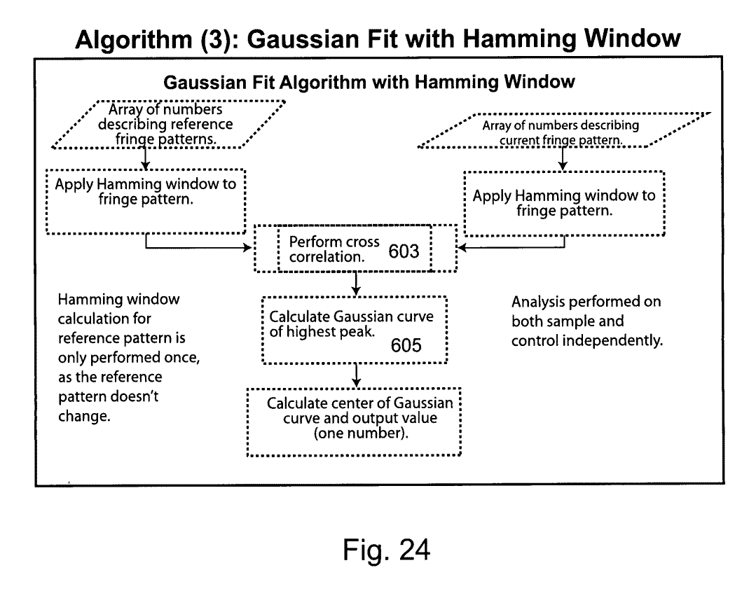

FIG. 24 shows the use of a hamming window, which is applied to the fringe pattern before cross correlation 603 is performed. Then Gaussian fit 605 of cross correlation 603 is used to determine the shift in the fringe pattern. The hamming window helps to minimize noise.

BSI can detect changes in refractive index in real time. Therefore, it is a useful tool for measuring binding assays in real time. Also, BSI can be used to compare two samples for differences in refractive index, thereby indicating differences between the contents of the two samples.

Interferometric detection is amenable to high throughput assay methods, as the molecules, particles or cells do not require labeling with other reagents, such as fluorescent tags, thus requiring less processing of individual samples. The presence of the mass of the immobilized target or a signal due to a binding pair in solution, in embodiments where no binding moiety is immobilized, is detected directly as a function of interferometric intensity and is robust under laser interrogation. The resulting signal is not susceptible to the photobleaching and loss of precision under long or repeated laser exposure of fluorescently labeled targets. Interferometric detection is a sensitive method of detection. Femtomolar levels of numbers of molecules can be detected and low picomolar (10-12) concentrations of target molecules can be detected.

An analyte in a sample can be detected in a sample in a number of ways. First, the interference patterns of a sample and a matched control can be compared. For example, a control sample should contain the same reagents and be contained in a container of the same dimensions as the test sample, but exclude the analyte. In this case, an important element that contributes to differences in the interference patterns will be differences in interaction between the analyte and the reagents in the two samples. For example, in a binding assay, differences between the concentration of an analyte between the two samples will be result in differences in amount of binding with a binding reagent, which, in turn, will result in differences in the interference pattern produced.

However, control and test samples may not be evenly matched. For example, a control plasma sample and a test plasma sample may have differences in various molecules that will result in differences in refractive index even if the concentrations of the analytes are the same. If analyte concentration differences contribute most to differences in refractive index, then this need not be an issue. However, these differences can be addressed in various ways. For example, a kit can provide reagents to construct a standard curve. Measuring results on the test sample against the standard curve provides an indication of the quantity of the analyte in the sample. Comparison of two samples, one with the reagents and one without, provides a measure of what contribution the presence of analytes make to changes in refractive index. A test sample can be divided between two containers, one with reagents and one without, for this purpose. Moreover, for heterogeneous assays which employ sample vessels for which capture molecules have been selectively deposited in given probe regions, sample and experimental measurements can be conveniently performed within a single tube. In this approach, sample of interest is selectively captured using capture molecules prudently localized within the probed region of the sample beam, while the reference beam interrogates a different region of the same vessel, which is devoid of extracted analyte. In this approach sample and reference measurements are performed on the sample matrix solution, variations in biological matrix, such as serological composition, ionic strength, and other bulk propertied can be compensated enhancing the signal to background.

The system can be used to determine the on- and off-kinetics of binding with a flowing system. In the flowing system, one molecule can be attached to the surface with chemistry. A running buffer is then flowed over the activated surface. Once the signal is stable, a second molecule that binds to the first is flown thought the system in increasing concentrations. When the sample interacts with the surface, there is an increase in signal until equilibrium is reached. When the running buffer is flowed back through, the bound molecules disassociate and the signal decreases and then equilibrates on the running buffer. For the reaction of the two molecules, an increase in signal is observed and then equilibrates. For this part of the curve, a ‘one phase exponential association’ equation is used [Y=Ymax*(1−exp(−K*X))] where K is the K observed. For the dissociation of the two molecules, a decrease in signal is observed until an equilibrium is reached. For this part of the curve, a ‘one phase exponential decay’ equation is used [Y=Span*exp(−K*X)+Plateau], where the K is the K off. The K on value is calculated by subtracting the K off from the K observed then dividing the value by the concentration of the binding ligand {Kon=(Kobs−Koff)/[ligand]}. The KD value is collected by dividing the K off by the K on [KD=Koff/Kon]. These equations assume one to one binding and that the concentration of one of the molecules is unchanged during the reaction. This is accomplished by the use of the flow as there is a constant amount of the same concentration being introduced into the channel.

5.6. Instrument with Continuous Injection

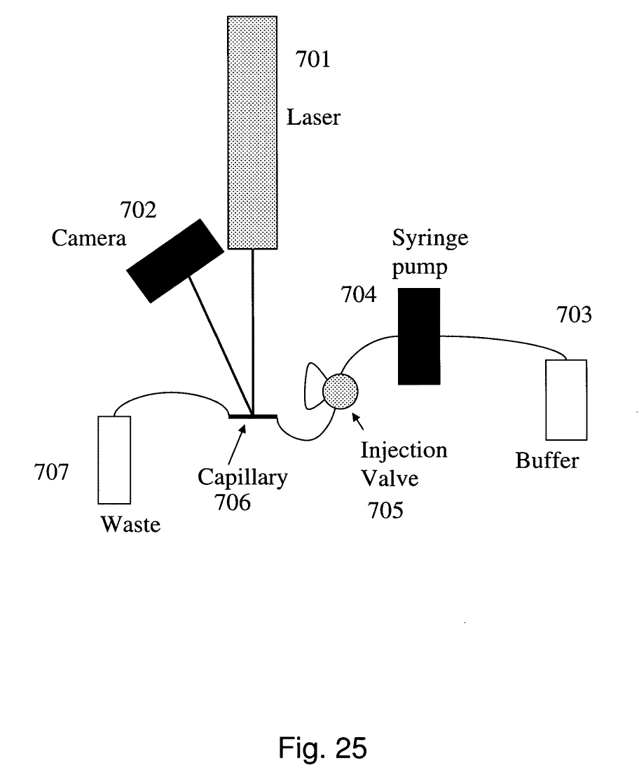

One version of the instrument allows for sample analysis in flowing streams. (See FIG. 25.) The basics of the instrumentation are the same; coherent light source 701 is directed onto fluidic channel 706, which produces a fringe pattern that is captured by camera 702.

Syringe pump (Cavro) 704 is utilized with an injection valve to create a flowing system. The syringe pump pulls in a volume of liquid from container 703 which is then dispensed at desired flow rates. These rates can range from 10 microliters per minute to 0.5 microliters per minute, e.g., approximately 2.5 μL/min. The fluid passes through an injection loop and then the detection zone of the instrument. This provides a continuous flow of running buffer in the system. The injection loop can have a volume of 20 μL that can be changed based on the size and length of tubing used. The injection valve 705 allows the injection of different samples without disrupting the flow of the system, as when in the load position the valve circumvents the loop allowing the running buffer to continuously flow. A sample is injected using a 250 μl analytical glass syringe into the loop. When the valve is switched to the inject position, the running buffer flows through the loop, pushing the injected sample into the detection zone. Thus the flow is never interrupted, aside from during the pump refill cycle.

The injected samples are pushed into the BSI instrument, which has a holder, which equilibrates the temperature of the fluid to a set point (typically 25° C.) by wrapping the capillary around a metal bobbin that is temperature controlled. The fluid is then pushed into the detection zone.

The detection zone is a small piece of capillary that the laser strikes. The small section of the capillary allows for surface chemistry to be performed on a large section and then cut into smaller sections for a heterogeneous experiment. After the fluid is analyzed, a waste tube is used to direct the sample into waste container 707.

### 6. Examples

### 6.1 Method of Producing a PEG Passivated Surface

The process is shown schematically in FIG. 1, and comprises steps of preparation of a cleaned surface, producing the passivated surface, and preparation for storage and future usage.

Preparation of a cleaned surface. A glass chip with one or more sample detection channels is sonicated in acetone for 5 minutes, where the channels of the chip are filled and flushed with solvent using a gentle vacuum. The acetone is removed, and the chip is sonicated for 5 minutes in methanol, using vacuum to fill the channels. The chip is then exposed to a solution of 99% sulfuric acid:30% hydrogen peroxide in a ratio of 4:1 v/v, filling the channels by vacuum, and maintained in the solution at 60° C. for 30 minutes. This solution is removed from the chip and its channels, and the chip and channels are rinsed twice with deionized water. The chip is then exposed to a solution of 28% ammonium hydroxide:30% hydrogen peroxide:deionized water in a ratio of 1:1:5 v/v, filling the channels. This ammonical solution is removed from the chip and its channels, and the chip and channels are rinsed twice with deionized water. The chip and its channels are then exposed to a solution of 37% hydrochloric acid:30% hydrogen peroxide:deionized water in a ratio of 1:1:5 v/v, filling the channels. This solution is removed from the chip and its channels, and the chip and channels are rinsed twice with deionized water. The chip is then dried under a nitrogen or argon stream, and further dried under vacuum at 120° C. overnight. A reaction vessel for the surface modification is also dried under the same vacuum conditions. The chip and reaction vessel are cooled under a nitrogen stream prior to removal from the vacuum oven.

Preparation of Passivated Surface. Anhydrous dimethyl formamide (18 ml) is added to the reaction vessel, and 2,4-toluene diisocyanate (2 ml) is added to the reaction vessel. The channels of the chip are filled manually under argon in a dry box with the reaction solution and the reaction is permitted to proceed for one hour. The chip and its channels are rinsed once, actively flushing the channels, with dimethylformamide. Dimethylformamide (18 ml), and PEG (2 ml) is added to a second reaction vessel in the dry box. The chip is placed into a second reaction vessel, the channels are filled manually, and the chip is maintained for one hour in the dry box as the PEG reacts with the isocyanate functionalities on the glass to form the PEG coupled brush monolayer. The covalent bond which attaches the PEG to the surface is a urethane bond.

Removal of excess reagent and storage. The chip is removed from the dry box and the channels are flushed twice with deionized water. The chip is dried under nitrogen and store under a dry atmosphere until ready for use.

### 6.2. PEG-200 Passivated Chips

Chips of several different materials, such as borofloat glass, aluminum, silicon and silicon nitride are treated as in Example 1 to introduce a PEG passivated surface, using PEG-200. The physical properties are determined for several of the chips.

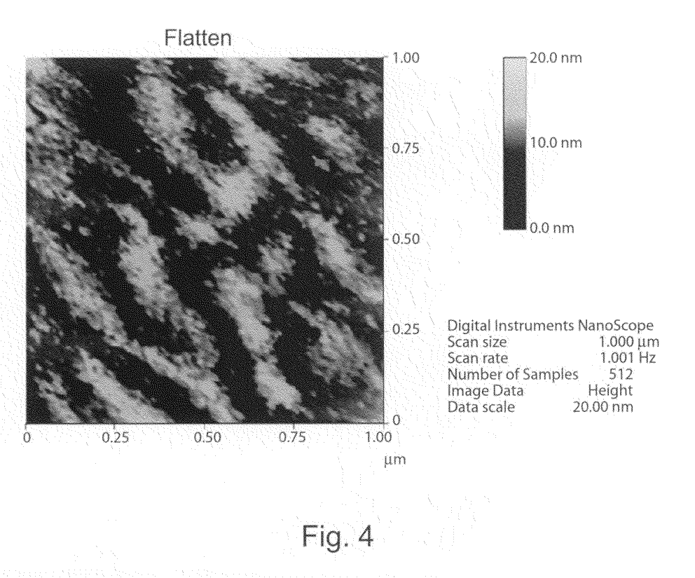

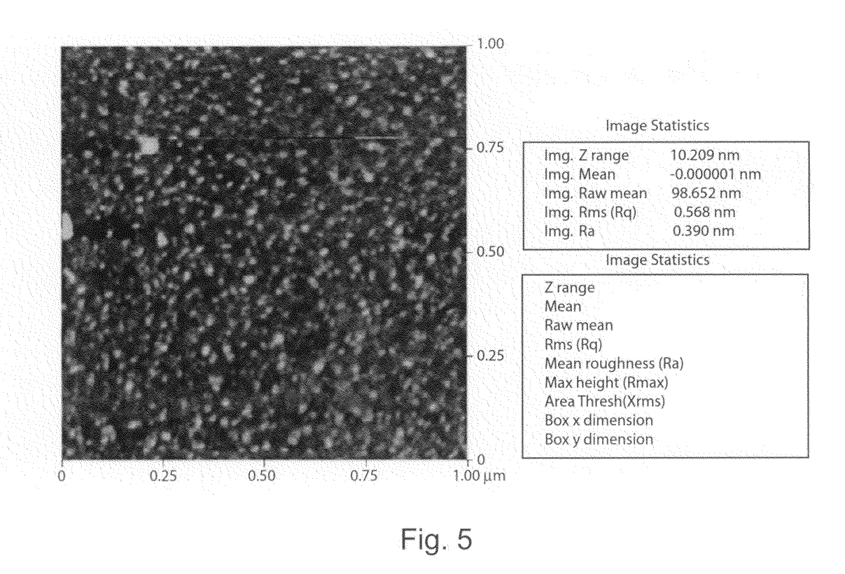

Atomic Force Microscopy (AFM, Digital Instrument Model SPM), in tapping mode using a silicon tip is used to determine the mean roughness Rₐ for two chips of differing substrate material but with a PEG-200 brush monolayer on each, as shown in FIG. 4 for a Borofloat 33 passivated chip and FIG. 5 for a silicon passivated chip. The images shown in FIG. 4 and FIG. 5 are scans of 1 μm×1 μm area. The Rₐ which is determined for the Borofloat 33 passivated chip is 1.4 nm, whereas the Rₐ determined for the PEG-200 brush passivated silicon wafer is 0.4 nm. Some of the difference in mean roughness shown is due to the original topology of the substrate; the silicon wafer of FIG. 5 possesses a more uniform native SiO₂ layer and hence yields a more uniform layer than that of the Borofloat 33 of FIG. 4.

Advance contact angle measurements are conducted using a Rame-hart instrument model 100-10, for a Borofloat 33 chip, a borofloat glass chip, and an aluminum chip, both uncoated and passivated. The contact angle measures surface tension and is a measure of the surface hydrophobicity/hydrophilicity, with a lower numerical value of the contact angle representing more hydrophilic surfaces.

TABLE 1
Contact Angle Measurements for Selected Chip Surfaces,
Unpassivated and Passivated

| | | |
|---|---|---|
| | Unpassivated Surface, | Passivated Surface, |
| Sample Identification | Contact Angle | Contact Angle |

| | | |
|---|---|---|
| MSI Borofloat 33 | 20 +/− 4° | 62 +/− 3° |
| glass chip | | |
| Borofloat 33 glass | 32 +/− 0.5° | 79 +/− 5° |
| slide | | |
| Alumina Witness | 85 +/− 5.2° | 71.8 +/− 1.5° |
| slide | | |

As is shown in Table 1, all of the test chips and slides show modulation of the contact angle upon passivation by PEG-200 brush monolayer. It is also of note, that with time the slides become hydrated and the contact angle decreases to some degree. (Data not shown.)

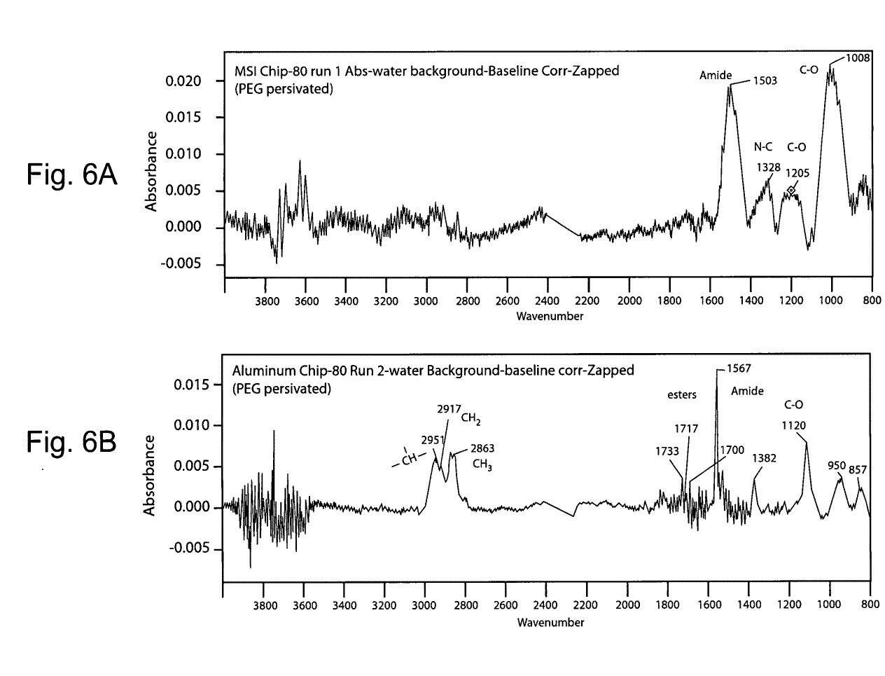

Fourier Transform Infrared Spectroscopy is performed using a BioRad FTIR, with a 85° grazing angle stage, on Borofloat 33, MSI, and Alumina passivated chips. Both the Borofloat 33 slide (Data not shown) and MSI passivated chips (FIG. 6A), exhibit C—O bond (1008 wavenumbers), C—N bond (1328), and amide bond (1503) absorptions in the spectra that is obtained. While the resolution of the spectrum is not ideal, identification of the critical peaks can be made to ascertain that the urethane bonds are present on the chips. The data for the alumina slide (FIG. 6B), shows all of the peaks as reported (C—O bond (1120 wavenumbers), amide (1567), C—N bond (1382)) for the glass chips, as well as ester (1777, 1733) and C—H bond (2951, 2917, and 2963) absorbance peaks.

Ellipsometry Characterization of the PEG-200 brush monolayer is performed using a spectroscopy ellipsometer (J. A. Woodlam). The Delta and Psi optical properties of the thin film on the substrate are measured, and compared against a Cauchy empirical model. The Cauchy equation is:

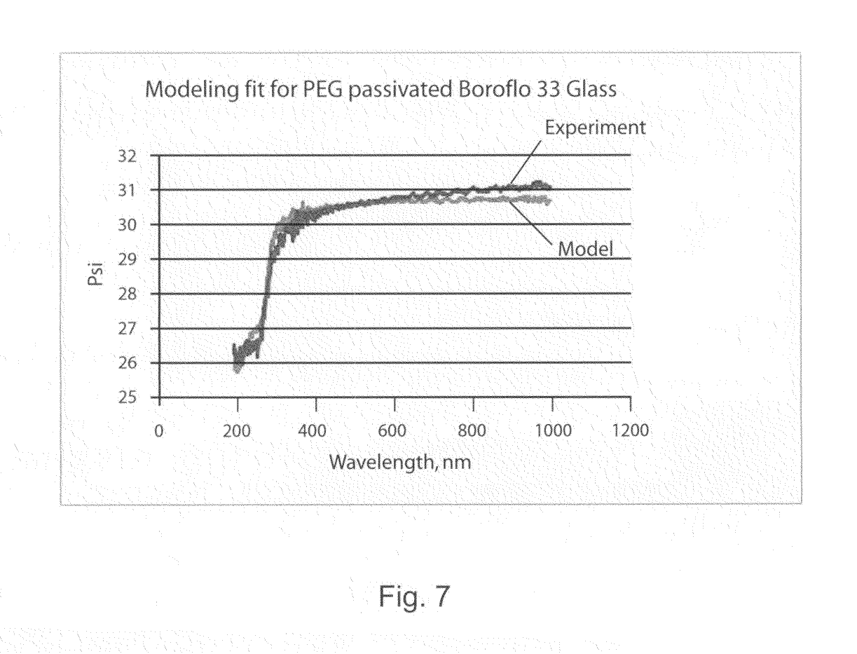

n(λ)=nₒ+n₁/λ²+n₂/λ² (Eq 4)

where n is the refractive index and λ is wave length. Based on the measurements and comparison against the model, it is determined that the PEG-200 brush film thickness is 3.1+/−0.4 nanometers, as shown in FIG. 7.

### 6.3. PEG-5mer Passivated Chip

Passivated chips using PEG-5mer in the brush monolayer are made using several substrate materials, according to the method of Example 1.

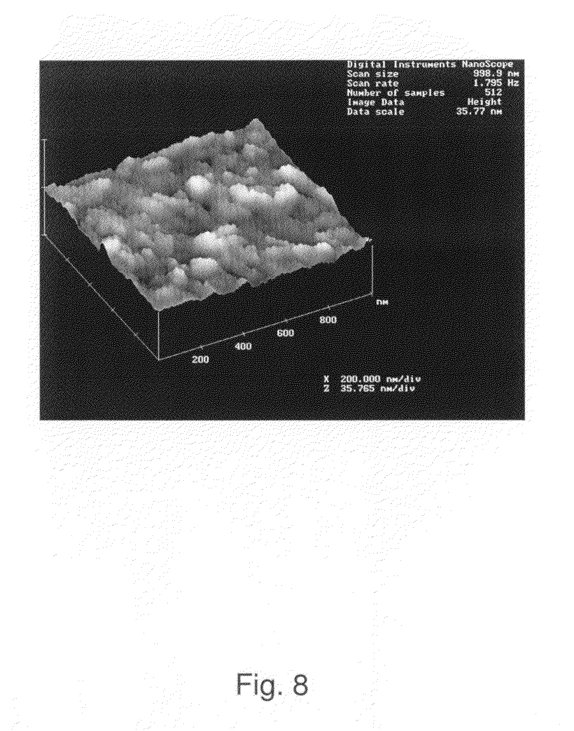

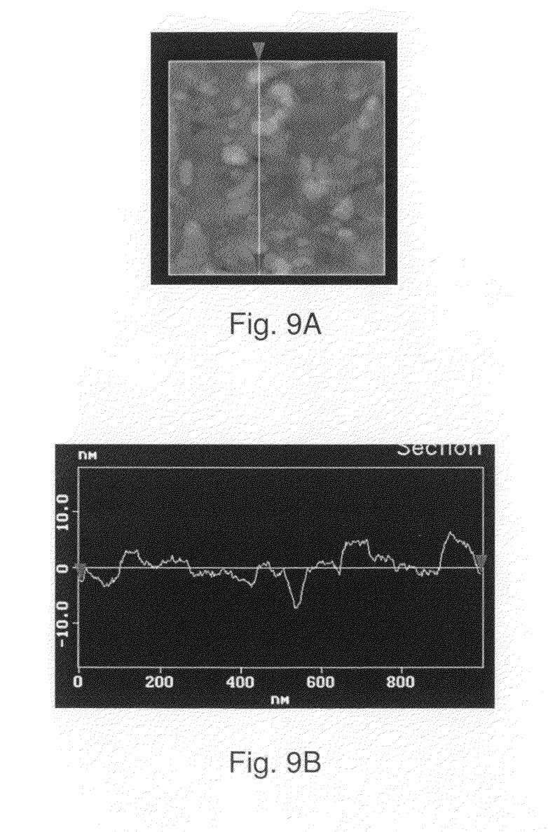

Atomic Force Microscopy is performed to determine the topology of the resultant passivated chips. FIG. 8 is a three dimensional representation of a scan of 1 μm×1 μm area of a silicon wafer passivated with PEG-5mer. A two dimensional representation of the roughness of the same chip is shown in FIG. 9A and the 1 dimensional roughness analysis is shown in FIG. 9B, with a mean roughness Rₐ of 1.7 nm.

Contact angle measurements are conducted for a Borofloat 33 chip and an alumina chip. In Table 2, the measured contact angles are represented. Borofloat 33 unpassivated surfaces have a contact angle of about 17°, which increases to 70.2° upon passivation with PEG-5mer. Conversely, alumina substrate has a contact angle of 85° when unpassivated and a reduced contact angle of 73.1° when passivated.

TABLE 2
Contact angle measurements for PEG-5 passivated chips.

| | Unpassivated Surface, | Passivated Surface, |
|---|---|---|
| Sample Identification | Contact Angle | Contact Angle |
| Borofloat 33 | 17.5 +/− 0.2° | 70.2 +/− 0.2° |
| Aluminum slide | 85.0° | 73.1 +/− 1° |

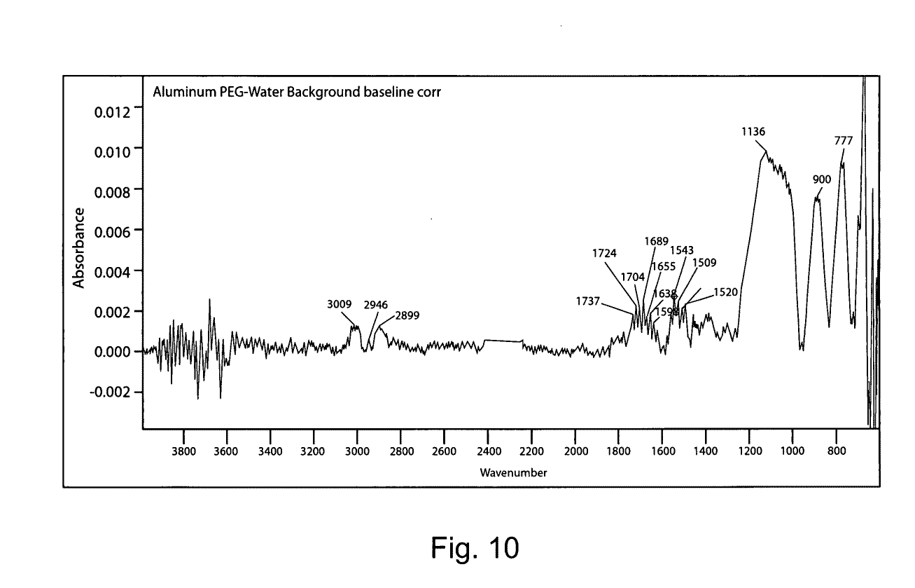

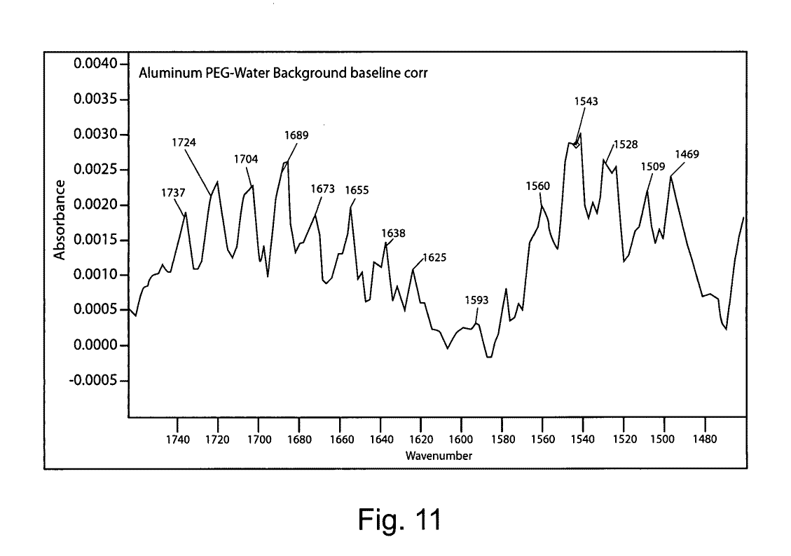

Fourier Transform Infrared Spectroscopy is performed and the results are shown in FIG. 10 and FIG. 11 for the alumina surface passivated with PEG-5mer. The presence of carbonyl, cyano and amide absorption peaks are present as seen in the case for the PEG-200 passivated surface, as above, and FIG. 11 shows an expanded region from 1400-1800 cm⁻¹, which shows a multitude of peaks for ester, C—N bond, and amide absorptions, clearly showing that the PEG immobilization through a urethane covalent bond is achieved.

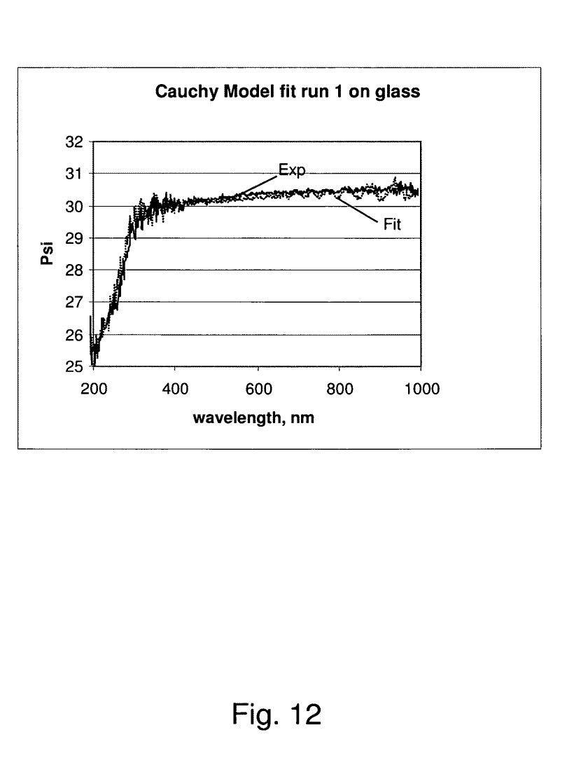

Ellipsometry Characterization of the PEG-5mer brush monolayer is performed, and the Delta and Psi values are obtained and compared against the Cauchy empirical model, which is shown in FIG. 12. The average film thickness on MSI borofloat chip is about 2.6+/−0.1 nm.

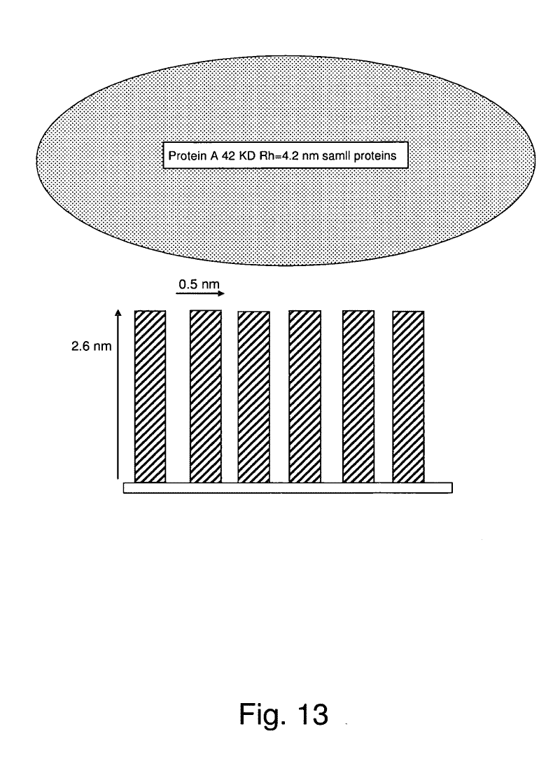

Density of the PEG-5mer polymer is 1.08 g/ml. The surface density is equal to density×thickness, which is 2.85×10⁻⁷ g/cm² or 6.91×10⁻¹² mol/mm². This is equivalent, dividing the number of moles per square millimeter by Avogadro's number, yielding 4.2 PEG-5mer chain attachments (bristles) per nanometer square. There is therefore, about 0.4 nanometers between each PEG-5mer chain connected to the surface. This is more dense than the equivalent silane attachments when a silanized surface is utilized, which is reported to be somewhere between 0.2 to 2 silane attachments per square nanometer. When taking into consideration the relative sizes of a potential protein of interest, for example, i.e., Protein A, which has a weight of 42 kDa, and a size of about 4.2 nm, cannot pass through the spacing of the PEG-5mer attachments to come into contact with substrate surface, as shown in FIG. 13, minimizing unwanted non-specific binding.

### 6.4. Epoxide Functionalized Passivated Chip

PEG-5mer preactivated chips are produced from the PEG-5mer passivated chip as described in Example 3. The process of producing the preactivated chips containing epoxide functionalities at the free termini of the PEG-5mer molecules in the brush monolayer is shown schematically in FIG. 3.

The channel of the PEG-5mer passivated borofloat glass chip is flushed with 20% phosgene in toluene in a fume hood for one hour. It is then flushed with toluene and dried under a nitrogen stream. The chip is transferred to a dry box and the channel(s) are flushed with 10% glycidol in DMF for 2 hours in the dry box. The reactive chloroformate groups react with glycidol to introduce terminal epoxy functional groups. The chip and its channel(s) are washed/flushed with methylene chloride and dried under a nitrogen stream. The resultant chip is stored under nitrogen atmosphere at reduced temperature (about 4-10° C.) before use.

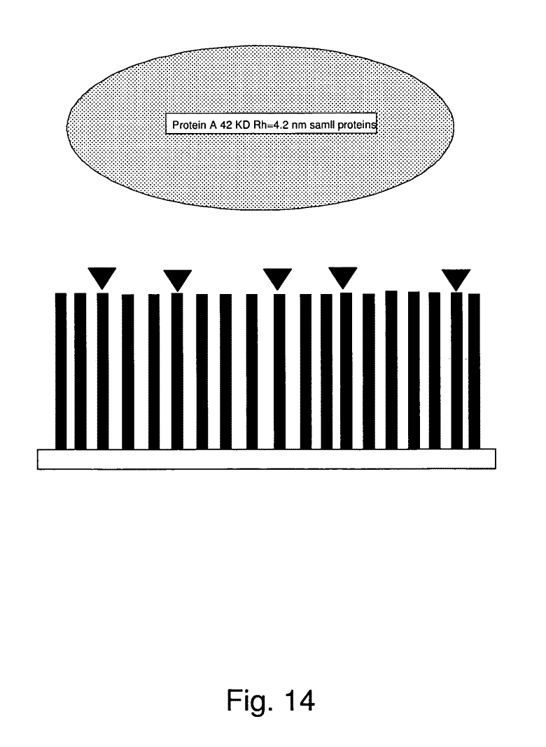

The epoxy chip is then exposed to a target molecule or binding moiety such as for example, IgG proteins such as Protein A, which is bound to the monolayer which is, in turn, bound to the substrate surface, while the protein is bound only to the epoxy groups, as shown in FIG. 14.

Contact angle measurements are determined for both Borofloat glass and Alumina PEG-5mer epoxy surfaces. As shown in Table 3, introduction of epoxy terminal groups further modulated the nature of the surface of the substrate that a target molecule contacts. The contact angle of the surface which is modified with epoxy may decrease with time, due to hydrolysis of the epoxy groups. The difference between the two substrates is due to the surface density of the PEG-5mer.

TABLE 3
Contact angle measurements for Borofloat 33
glass and Alumina slide

| | Unpassivated | | |
|---|---|---|---|
| Sample | Surface, | Passivated Surface, | Epoxidized Surface, |
| Identification | Contact Angle | Contact Angle | Contact Angle |
| Borofloat 33 | 17.5 +/− 0.2° | 70.2 +/− 0.2° | 66 +/− 0.9° |
| Aluminum | 85.0° | 73.1 +/− 1° | 53.5 +/− −.2° |

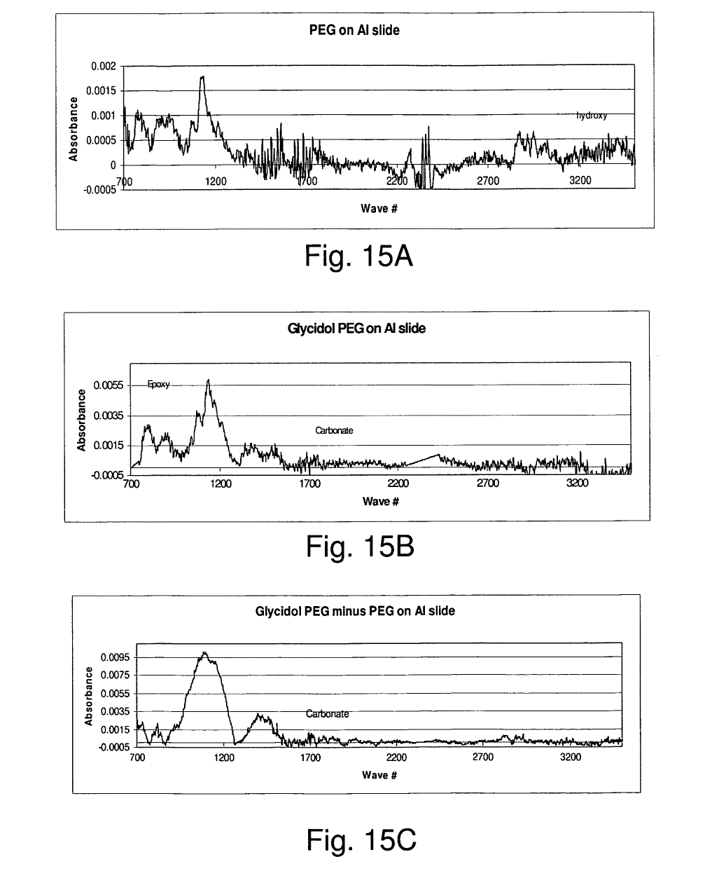

Fourier Transform Infrared Spectroscopy is performed on these epoxy activated PEG-5mer passivated surfaces. In FIGS. 15A, 15 B, and 15 C are shown a series of FTIR spectra for an aluminum PEG-5mer passivated surface. Spectrum A shows the absorptions due to the PEG-5mer passivated monolayer, as for Example 3. Spectrum B shows the spectrum due to the combination of the PEG-5mer passivated monolayer with epoxy functionality introduction. Spectrum C shows a subtraction of Spectrum A from Spectrum B to give the spectrum of infrared absorbance due to the epoxy functionality introduction itself, and is clearly seen in the peaks at 800-1200 cm⁻¹.

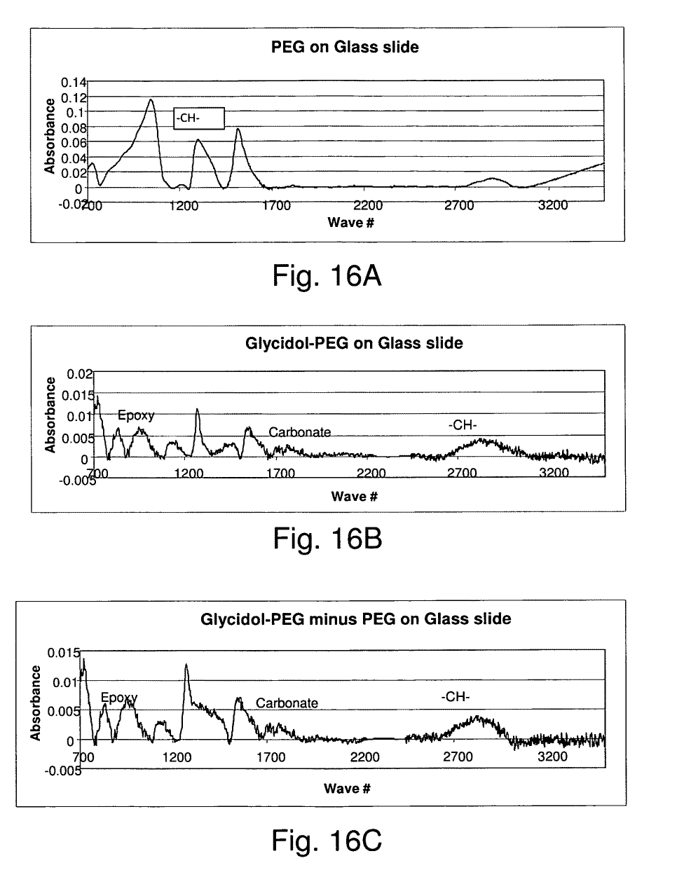

The corresponding sequence of FUR spectra is shown in FIGS. 16 A-C for a borofloat glass chip The resolution is poorer on the glass substrate but for FIG. 16A, carbon-oxygen single bond absorbance is seen at 1100 wavenumbers, carbon-hydrogen single bond absorbance at 1300, and urethane bond absorbance is at 1550-1650. After introduction of the epoxy groups, the modified substrate has a spectrum as shown in FIG. 16B, with epoxy absorbance peaks in the region of 800-990 wavenumbers. Subtracting out the spectrum (FIG. 16-A) for the PEG-5mer passivated glass surface, FIG. 16C represents the FTIR spectra due to the epoxy functionalities introduced on this surface, with epoxy absorbance peaks at 800-990 cm⁻¹ and carbonate absorbance at 1650 cm⁻¹.

### Example 5

### Determination of Reduction of Nonspecific Binding for a PEG-200 Passivated Glass Surface

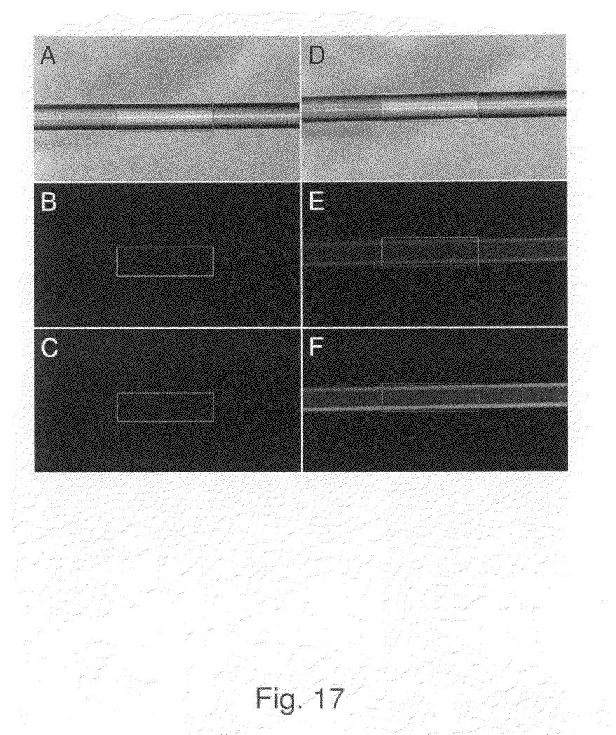

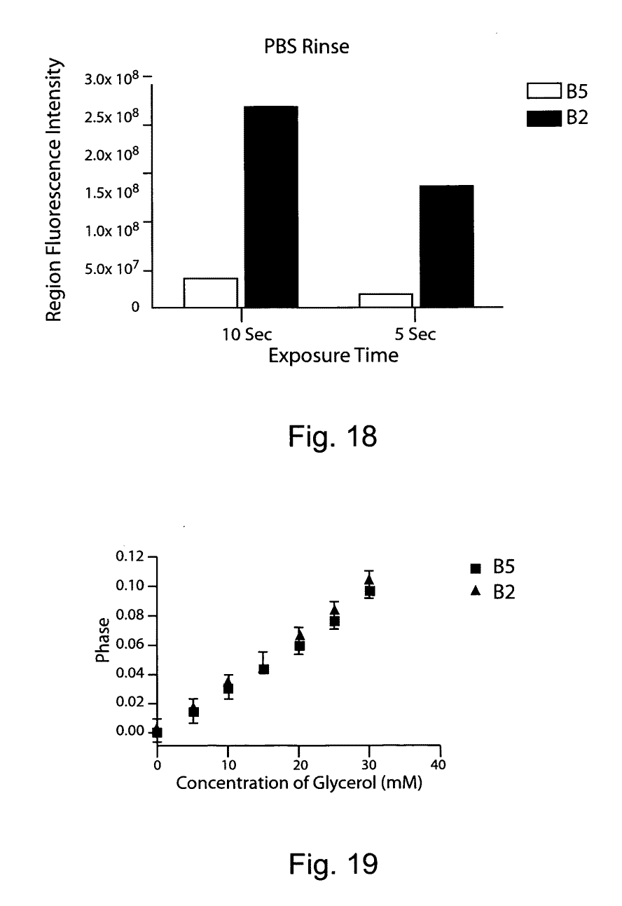

Using a channeled glass chip, the extent of nonspecific binding of a fluorescently labeled protein is determined for both an unmodified glass channel surface and a PEG-200 passivated channel surface of a glass substrate of the same type. As shown in FIG. 17, under white light illumination, panel A shows a channel B5 with a PEG-200 passivated surface and panel D shows a channel B2 with no treatment, which has unmodified glass surfaces. The center section was used to collect quantitative fluorescent data. The channels of both unmodified and PEG-200 passivated substrates (Panels A and C, respectively) are filled with 1 mg/ml fluorescent Protein A-FITC and incubated at room temperature for 10 minutes. After flushing with deionized water (10 μl) and then PBS, both channels are imaged using fluorescence microscopy. The unmodified channel B2 shown in panels D, E, and F, exhibit higher levels of fluorescence, and hence nonspecific binding as compared to the PEG-200 passivated channel B5 shown in panels A, B, and C. The intensity is quantified using a same size region analysis of the images and is shown in FIG. 18, and demonstrates an 88% reduction in nonspecific binding after a 10 minute incubation with a hydrophobic material like protein A FITC.

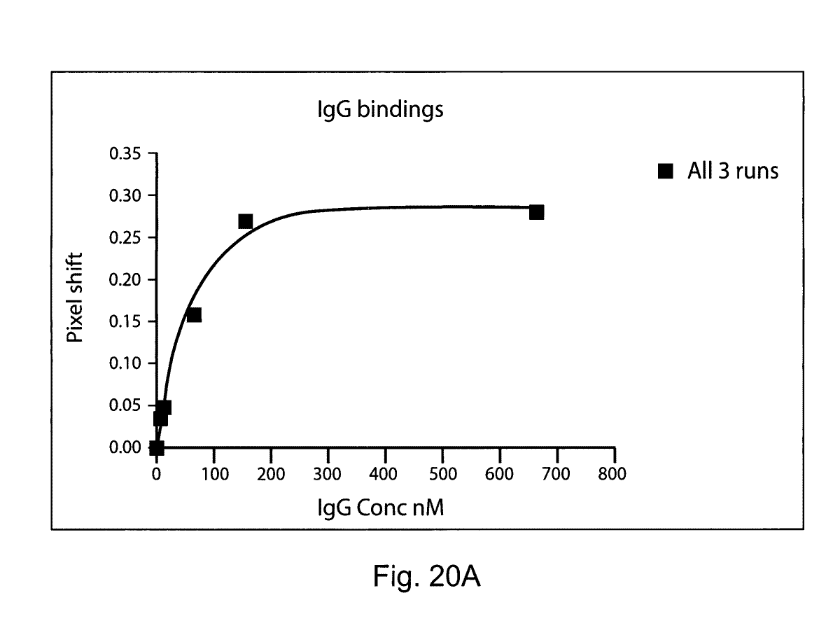

Performance evaluation of B2 and B5 by glycerol calibration under back scattering interferometric conditions. Channels of both unmodified and modified substrates are filled with increasing concentrations of glycerol (x axis) and evaluated for alterations due to the presence of the monolayer. Back scattering pixel shift as a function of the glycerol concentration is recorded as the y-axis. The calibration curves are shown in FIG. 19 for chips B2 and B5. The slopes (sensitivity) are very similar for both chips and the 3σ detection limits are comparable as shown in Table 4. The correlation of the two curves indicates that the introduction of the PEG monolayer does not change the back scattering observed and therefore does not affect the sensitivity of the detection of the instrument used.

TABLE 4
Detection limits of the glycerol calibration for B5 and B2 chips.

| | B5 | B2 |
|---|---|---|
| Limit of detection | 3.90 mM = | 2.28 mM = 2.28 × 10⁻⁵ RIU |
| | 3.90 × 10⁻⁵ RIU | |

### 6.5. Determination of Biological Binding Affinities Using the Passivated Chips of the Invention

The PEG-200 and PEG-5mer passivated chips of the invention were used to determine the binding constant KD of the IgG-Protein A binding interaction, using Back Scattering Interferometry. Protein A-IgG samples are prepared in PBS in the following concentrations: All samples contains 0.67 μM of Protein A and IgG concentrations are from varied from 0, 1.5, 6.7, 13.4, 67, to 154 μM to give 6 concentration data points. Each sample is incubated at room temperature for 2 hours. These samples are injected into the PEG 200 and PEG-5mer passivated chips, and the extent of binding is measured by back scattering interferometric detection of pixel shift, which yields the change of refractive index for each triplicated data point.

The equilibrium constant is derived below:

Protein A+IgG-→Protein A-IgG (Eq 5)

At t=0 after mixing, the protein A concentration is C, IgG is P, and the protein A-IgG concentration is zero At time t, the protein A concentration is still C (excess) and IgG is (1−θ)P and protein A-IgG complex is θP where θ is the fraction of reaction completion.

The forward (association) rate=ka*P(1−θ) (Eq. 6)

The backward (dissociation) rate=kd*θ*P (Eq. 7)

At equilibrium, Ka*P*(1−θ)=kd*P*θ (Eq. 8)

θ=P/(KD+P) where: (Eq. 9)

Dissociation constant KD=kd/ka (Eq. 10)

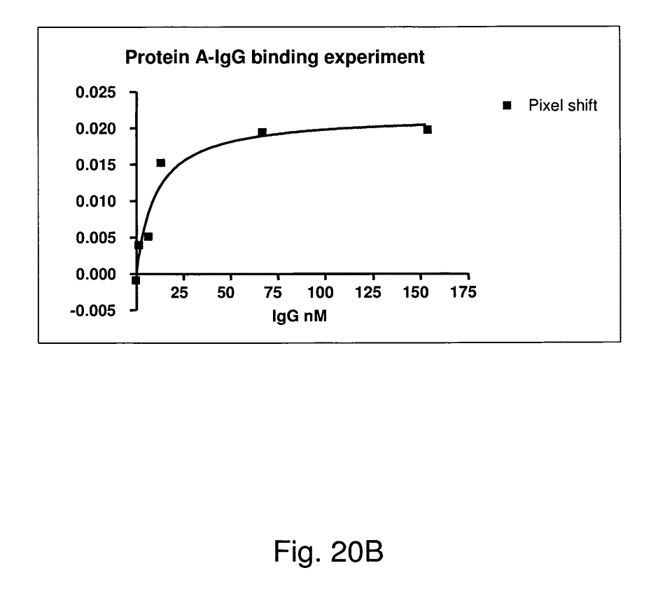

When θ=0.5, KD=P, which represent the IgG concentration at 50% completion of binding. The data from these experiments are shown in the curves in FIG. 20A for a IgG Fc segment on a PEG 200 passivated chip and FIG. 20B for IgG on a PEG-5mer passivated chip, and yield a KD determination values of 35 nM and 8 nM respectively.

### REFERENCES

U.S. Pat. No. 4,093,759 OTSUKI et al. Jun. 6, 1978

U.S. Pat. No. 4,268,554 GRAS May 19, 1981

U.S. Pat. No. 5,325,170 BORNHOP Jun. 28, 1994

U.S. Pat. No. 6,381,025 BORNHOP et al. Apr. 30, 2002

U.S. Pat. No. 6,809,828 BORNHOP et al. Oct. 26, 2004

U.S. Pat. No. 7,130,060 BORNHOP et al. Oct. 31, 2006

International application WO 2004/023115 BORNHOP et al. Mar. 18, 2004

International application WO 2006/047408 BORNHOP et al. May 4, 2006

International application WO 2008/144496 JONES et al. Nov. 27, 2008

International application WO 2009/039466 BORNHOP Mar. 26, 2009

U.S. 2004-0115721 MAO et al. Jun. 17, 2004

U.S. 2008-0160187 MURATA et al. Jul. 3, 2008

U.S. 2008-0194723 HUANG et al. Aug. 14, 2008

U.S. 2006-0012800 BORNHOP et al. Jan. 29, 2006

U.S. 2009-0185190 WEINBERGER et al. Jul. 23, 2009

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An article comprising a substrate comprising a surface, and a PEG monolayer attached to the surface through a covalent linkage, wherein a plurality of PEG moieties comprise terminal hydroxy groups.

2. The article of claim 1 wherein the surface is glass, silicon, silicone, polymer, ceramic, metal-oxide or metallic.

3. The article of claim 1 wherein the PEG monolayer comprising terminal hydroxy groups is a brush monolayer.

4. The article of claim 1 wherein the PEG monolayer comprising terminal hydroxy groups is a self assembled monolayer (SAM).

5. The article of claim 1 wherein the covalent linkage is a urethane linkage.

6. The article of claim 1 wherein the PEG is PEG 2-50mer.

7. The article of claim 1 wherein the PEG is PEG 2-20mer.

8. The article of claim 1 wherein the PEG is substantially monodispersed.

9. The article of claim 1 wherein the PEG is at least 95% monodispersed.

10. The article of claim 1 wherein the PEG is PEG-200.

11. The article of claim 1 wherein the PEG is PEG 5mer.

12. The article of claim 1 wherein a plurality of PEG moieties comprise a reactive functionality coupled through a terminal hydroxy group.

13. The article of claim 12 wherein the reactive functionality is an epoxide, NHS, NHM, imidazolecarbonyl, hydrazine, aldehyde, iodoacetyl, cystamine, or DTT group.

14. The article of claim 12 wherein the reactive functionality is further coupled to a binding moiety.

15. The article of claim 14 wherein the binding moiety is a biomolecule.

16. The article of claim 15 wherein the biomolecule is an antibody, nucleic acid, organic molecule, protein, particle, or cell.

17. The article of claim 16 wherein the organic molecule is a hapten, receptor ligand, phosphatidyl glycol, avidin, biotin, organometallic, drug molecule, aptamer, or heparin.

18. The article of claim 1 which is a capillary tube, a pipette tip, a microtube, a microwell, a microtiter plate, an array with addressable sites, a waveguide, a compact disc, or a chips with continuous lanes or contiguous surfaces.

19. An article comprising a substrate comprising at least one channel having a surface wherein PEG is attached to the surface as a monolayer and a plurality of PEG moieties comprise a free terminal hydroxy group.

20. The article of claim 17 wherein the surface is glass, silicon, polymer, ceramic, metallic oxide or metallic.

21. The article of claim 17 wherein the substrate comprises two pieces sandwiched together, wherein at least one piece comprises a groove that defines the channel in the sandwich.

22. The article of claim 17 wherein the channel is a microfluidic channel having a diameter no greater than 500 microns.

23. The article of claim 17 wherein the channel is a microfluidic channel having an average cross sectional area of about 0.2 mm².

24. The article of claim 17 wherein the at least one channel is at least two channels.

25. The article of claim 17 wherein the PEG is PEG 2-50mer.

26. The article of claim 17 wherein the PEG is monodispersed.

27. The article of claim 17 wherein the PEG is PEG 2-20mer.

28. The article of claim 17 wherein the PEG is PEG-200.

29. The article of claim 17 wherein the PEG is PEG 5mer.

30. The article of claim 17 wherein a plurality of terminal hydroxy groups of the PEG are coupled to a reactive functionality.

31. The article of claim 30 wherein the reactive functionality is an epoxide, NHS, NHM, imidazolecarbonyl, hydrazine, aldehyde, iodoacetyl, Cystamine, or DTT group.

32. The article of claim 30 wherein the reactive functionality is further coupled to a binding moiety.

33. The article of claim 32 wherein the binding moiety is coupled through reaction with the reactive functionality.

34. The article of claim 32 wherein the binding moiety is a biomolecule.

35. The article of claim 34 wherein the biomolecule is an antibody, nucleic acid, organic molecule, protein, particle, or cell.

36. The article of claim 35 wherein the organic molecule is a hapten, receptor ligand, phosphatidyl glycol, avidin, biotin, organometallic, drug molecule, aptamer, or heparin.

37. An article comprising a channel having a passivated surface comprising terminal hydroxy groups that exhibits substantially no nonspecific binding.

38. The article of claim 37 wherein the channel is a microfluidic channel.

39. The article of claim 37 wherein the surface is glass, silicon, silicone, polymer, ceramic, metal-oxide or metallic.

40. The article of claim 37 wherein the passivated surface exhibits at least 95% reduction of nonspecific binding compared to a nonpassivated surface.

41. The article of claim 37 wherein the surface is passivated with a monolayer of PEG comprising terminal hydroxy groups.

42. The article of claim 42 wherein the monolayer of PEG is a brush monolayer.

43. The article of claim 41 wherein the monolayer is a self assembled monolayer (SAM).

44. An article comprising a substrate having a microfluidic channel having a passivated surface comprising terminal hydroxy groups wherein the surface comprises a monolayer of a polymer of no more than 20 monomeric units.

45. The article of claim 44 wherein the surface is glass, silicon, silicone, polymer, ceramic, metal-oxide or metallic.

46. The article of claim 44 wherein the monolayer is no more than 100 Ångstroms thick.

47. The article of claim 46 wherein the polymer is covalently attached to the surface.

48. The article of claim 44 wherein the polymer is PEG.

49. The article of claim 48 wherein the PEG is PEG-200.

50. The article of claim 48 wherein the PEG is PEG 5mer.

51. A method of passivating a surface comprising:
(a) reacting the surface with a reagent bearing reactive groups at both termini to produce the modified surface having a terminal reactive group; and
(b) reacting PEG comprising two terminal hydroxy groups with the terminal reactive group to attach PEG to the surface through a covalent bond to form a passivated surface comprising PEG which comprises terminal hydroxy groups.

52. The method of claim 51 wherein the surface is glass, silicon, polymer ceramic, metallic oxide or metallic.

53. The method of claim 51 wherein the reagent bearing reactive groups at both termini is toluene diisocyanate.

54. The method of claim 51 wherein the terminal reactive group is an isocyanate group.

55. The method of claim 51 wherein the covalent bond is a urethane bond.

56. The method of claim 51 wherein the PEG is PEG 2-50mer.

57. The method of claim 56 wherein the PEG is PEG-200.

58. The method of claim 56 wherein the PEG is PEG 5mer.

59. A method of manufacturing an article comprising two pieces sandwiched together, wherein at least one piece comprises a groove that defines the channel in the sandwich and wherein the two pieces each comprise a surface comprising a PEG monolayer comprising terminal hydroxy groups attached to the surface through a covalent linkage, comprising:
(a) attaching a reactive functionality to at least one surface comprising a PEG monolayer which comprises terminal hydroxy groups, and
(b) bonding both surfaces together by reacting the functionality to the PEG monolayer comprising terminal hydroxy groups on the surface of the second piece.

60. The method of claim 59 wherein the bonding is at room temperature.

61. The method of claim 59 wherein the reactive functionality is epoxide, acrylate, or methacrylate.

62. A method comprising:
(a) providing a substrate comprising a glass surface having a PEG monolayer comprising terminal hydroxy groups attached thereto, wherein a portion of the terminal hydroxy groups of the PEG comprises terminal reactive functionalities; and
(b) reacting the terminal reactive functionalities with binding moieties to couple the binding moieties to the surface.

63. The method of claim 62 wherein the terminal reactive functionalities are epoxide, NHS, NHM, imidazolecarbonyl, hydrazine, aldehyde, iodoacetyl, Cystamine, or DTT groups.

64. The method of claim 62 wherein the binding moiety is a biomolecule.

65. The article of claim 64 wherein the biomolecule is an antibody, nucleic acid, organic molecule, protein, particle, or cell.

66. The article of claim 53 wherein the organic molecule is a hapten, receptor ligand, phosphatidyl glycol, avidin, biotin, organometallic, drug molecule, or heparin.

67. A method comprising:
(a) providing a chip comprising a microfluidic channel therein defined by a wall, wherein the wall is passivated with a monolayer of PEG which comprises terminal hydroxy groups;
(b) flowing a fluid through the channel, wherein the fluid comprises a pair of binding partners; and
(c) detecting binding of the binding partners in the channel.

68. The method of claim 67 wherein binding is detected by interferometry.

69. A method comprising:
(a) providing a chip comprising a channel therein defined by a wall, wherein the wall is passivated with a monolayer of PEG comprising terminal hydroxy groups and comprising a binding moiety attached to the wall;
(b) flowing a fluid through the channel, wherein the fluid comprises a binding partner for the binding moiety; and
(c) capturing the binding partner with the binding moiety.

70. The method of claim 69 wherein the channel is a microfluidic channel.

71. The method of claim 69 wherein the binding moiety is a biomolecule.

72. The method of claim 70 wherein the biomolecule is an antibody, nucleic acid, organic molecule, protein, particle, or cell.

73. The method of claim 72 wherein the organic molecule is a hapten, receptor ligand, phosphatidyl glycol, avidin, biotin, organometallic, drug molecule, or heparin.

74. The method of claim 69 further comprising detecting binding of the binding partner in the channel.

75. The method of claim 70 wherein binding is detected by interferometry.

76. The method of claim 67 wherein the effective concentration of detectable binding of the binding partners is lower than 100 pM.

77. The method of claim 74 wherein the effective concentration of detectable binding of the binding partners is lower than 100 pM.
